(19) United States
(12) Patent Application Publication
(10) Pub. No.: US 2012/0329879 A1
(43) Pub. Date: Dec. 27, 2012

(54) BETA-ADRENERGIC RECEPTOR AGONISTS AND USES THEREOF

(75) Inventors: Jena J. Steinle, Olive Branch, MS (US); Kimberly P. Williams, Memphis, TN (US); Jayaprakash Pagadala, Memphis, TN (US); Duane D. Miller, Germantown, TN (US)

(73) Assignee: THE UNIVERSITY OF TENNESSEE RESEARCH FOUNDATION, Knoxville, TN (US)

(21) Appl. No.: 13/607,216

(22) Filed: Sep. 7, 2012

Related U.S. Application Data
(63) Continuation-in-part of application No. PCT/US2011/000428, filed on Mar. 8, 2011.
(60) Provisional application No. 61/339,679, filed on Mar. 8, 2010.

(51) Int. Cl. A61K 31/137; C07C 215/56; C12N 5/071; A61P 27/02

(52) U.S. Cl. 514/653; 564/361; 435/375

(57) ABSTRACT

Provided herein are methods for improving function in a retinal cell associated with a diabetic condition and for treating a diabetic retinopathic condition in a subject. The methods comprise contacting the retinal cell or administering to the subject a beta-adrenergic receptor agonist or R-isomer thereof such as have the chemical structural formula:

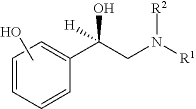

where R¹ is (CH₂)ₙ(CH₃)₂ or

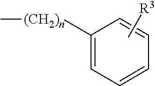

where n is 1 to 4, R² is H or H.HX, where X is a halide and R³ is O(CH₂)ₘCH₃ at one or more of C2-C6, where m is 0 to 4. Also provided are BAR agonists having the structural where R¹ is the (CH₂)ₙ-phenyl-R² substituent and the hydroxy-benzene moiety is 1,2-benzene diol or 1,3-benzene diol.

### CROSS-REFERENCE TO RELATED APPLICATION

This is a continuation-in-part application under 35 U.S.C. §120 of pending international application PCT/US2011/000428, filed Mar. 8, 2011, which claims benefit of priority under 35 U.S.C. §119(e) of provisional application U.S. Ser. No. 61/339,679, filed Mar. 8, 2010, now abandoned, the entirety of both of which are hereby incorporated by reference.

### BACKGROUND OF THE INVENTION

1. Field of the Invention

The present invention relates to the fields of diabetes and eye disease. Specifically, the present invention provides compounds and methods for treating pre-proliferative diabetic retinopathy.

2. Description of the Related Art

Diabetic retinopathy is the leading cause of blindness in working age adults. Nearly all diabetics show some signs of retinopathy within 20 years of diagnosis. The cost to the US in health care for diabetic patients was $174 billion in 2007 alone. Major hallmarks of human diabetic retinopathy, as well as animal models of the disease; include increased glial inflammatory markers and neuronal cell death that results in vision loss. While insulin therapy can slow the overall progression of the disease, mechanisms of insulin regulation in the retina remain unclear and there is no targeted treatment to prevent vision loss.

Minimal treatments for diabetic retinopathy have been put into clinical use since the 1970's, none designed to target pre-proliferative diabetic retinopathy. The current treatment for the proliferative phase of diabetic retinopathy is laser photocoagulation, which is effective in the late phases of proliferative diabetic retinopathy. Many diabetic and hypertensive patients are placed on beta-adrenergic receptor antagonists and this is effective at blood pressure reduction. However, there has been no thorough analysis of effects of these agents on the human retina.

One report in rodents suggested that the beta-adrenergic receptor antagonists had little effect on the retina (1). In contrast, other studies using a beta-adrenergic receptor antagonist given systemically to rodents, demonstrated that propranolol, a commonly used beta-adrenergic receptor antagonist, produced significant deficits in the electrical activity in the retina and activated growth factors that may promote neovascularization (2).

Inflammatory mediators are key factors in diabetic retinopathy. Insulin receptor signaling is triggered by the release of insulin. Beta-adrenergic receptors modulated protein levels of both inflammatory mediators and insulin signaling. Particularly, TNFalpha levels are reduced by beta-adrenergic receptor agonists, in multiple cell types of the retina.

There are limited approaches to treatment pre-proliferative phase of diabetic retinopathy, which occurs before vascular damage develops. However, this is the most suitable phase for treatment since vision could theoretically be weakened prior to development of permanent blindness. Numerous hypotheses have been offered to explain the retinal pathologies associated with hyperglycemia, yet none has been translated to patient care for the pre-proliferative phase of the disease. It seems reasonable to identify biological markers reflecting early stages of the disease development, so that treatment can be initiated prior to irreversible vascular damage.

Therefore the prior art is deficient in effective methods and tools for treatment of the pre-proliferative diabetic retinopathy. The present invention fulfills this longstanding need and desire in the art.

### SUMMARY OF THE INVENTION

The present invention is directed to a method for improving function in a retinal cell associated with a diabetic condition. The method comprises contacting the cell with a beta-adrenergic receptor (BAR) agonist, where the beta-adrenergic receptor agonist increases insulin signaling and insulin-like growth factor binding protein-3 (IGFBP-3) and decreases TNFalpha-induced apoptosis, thereby improving the function in the retinal cell. The beta-adrenergic receptor agonists may have the general chemical structure or may be the R-isomer thereof:

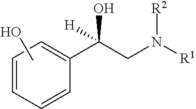

where R¹ is (CH₂)ₙ(CH₃)₂ or

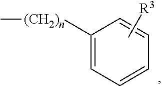

n is 1 to 4, R² is H or H.HX, where X is a halide, and R³ is O(CH₂)ₘCH₃ at one or more of C2-C6, where m is 0 to 4.

The present invention also is directed to a method for treating a diabetic retinopathic condition in a subject. The method comprises administering one or more times a pharmacologically effective amount of one or more beta-adrenergic receptor agonists or a pharmaceutical composition thereof to the subject, where the agonist improves retinal cell function, thereby treating the diabetic retinopathy. The present invention is directed to a related method of further comprising administering of one or more other diabetic or retinopathic drugs to the subject. The beta-adrenergic receptor agonists may have the general chemical structure or may be the R-isomer thereof, as described herein.

The present invention is directed further to a beta-adrenergic receptor agonist having the chemical structural formula or a pharmaceutical composition thereof:

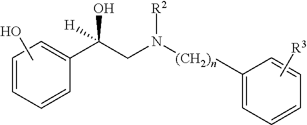

where n is 1 to 4, R² is H or H.HX, where X is a halide, and R³ is O(CH₂)ₘCH₃ at one or more of C2-C6, where m is 0 to 4.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention as well as others which will become clear are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1 illustrate the effects of Compound 2 on PKA activity and CREB phosphorylation, and demonstrates that 1 mM given daily to diabetic rats this compound significantly increased PKA activity in the retina as compared to 10 mM and with no effect on the control rats (P<0.05, vs. Ctrl and Diab+2, N=5). This data shows that topical administration of Compound 2 reaches the retina and initiates normal cellular signaling.

FIGS. 2A-2I depict waveforms. Representative waveform from 1 animal in each of the groups were recorded using ERG (FIGS. 2A-2C) or OP (FIGS. 2D-2F). Line graphs with the means and standard deviation for all the animals in each group are shown at the increasing light intensities for the a-wave (FIG. 2G), b-wave (FIG. 2H) and oscillatory potentials (FIG. 2I) recorded using ERG. It is clear that topical Compound 2 can inhibit the loss of all three components of the ERG over the entire 8-month period. Error bars are mean SD. A-wave, B-wave amplitude and OCT amplitude were measured monthly in each group via electroretinogram (ERG) analysis. Data is presented for animals at 2, 6, and 8 months of diabetes. While there was little difference seen between ERG amplitudes of control rats and those receiving 1 mM Compound 2 at 2 and 8-months, diabetic only rats showed a significant reduction in a-wave, b-wave amplitude and oscillatory potential amplitudes (FIGS. 2A-2E, P<0.05 vs. Ctrl and Diab+2, N=6). Results indicate that Compound 2 treatment was able to maintain normal electric activity in the retina throughout the experiment.

FIGS. 3A-3F compare the central and peripheral retinal thickness and number of cells in the ganglion cell layer in control rats, diabetic rats and diabetic rats plus Compound 2 and image the photoreceptor cell bodies, the bipolar cells and ganglion cell layers where Compound 2 is administered as a preventative (FIGS. 3A-3C) and as delayed treatment (FIG. 3D-3F). The image for diabetic rats is shorter, since the inner retinal thickness is reduced. It has been demonstrated that diabetes decreases cell number and retinal thickness at 2 months (Jiang et al, 2010). In both the peripheral and central retina, the thickness of the retina was significantly reduced in diabetic rats receiving no treatment. The cell number in the ganglion cell layer (GCL) of the peripheral and central retinas were significantly reduced in diabetic rats as compared to control or diabetic+2 animals (P<0.05 vs. Ctrl and Diab+2, N=5). Treatment with Compound 2 maintains the retinal thickness and cell number in spite of diabetes in the retina.

FIGS. 4A-4B show the effect of Compound 2 in the eye. FIG. 4A shows the number of degenerate capillaries per square millimeter of retina (P<0.05 vs. Ctrl and Diab+2, N=4). Eye drop treatment significantly reduced numbers of degenerate capillaries in diabetic rats. The number of Pericyte ghosts per 1,000 capillaries is shown in FIG. 4B (P<0.05 vs. Ctrl and Diab+2, N=4).

FIGS. 5A-5D shows that Compound 2 significantly reduced levels of TNFalpha activity in vitro. The same compound was examined in vivo as causing the decrease of inflammatory marker levels in diabetic rats. FIGS. 5A-5B show of TNFalpha activity in the retina at 2-months (FIGS. 5A-5B) (P<0.05 vs. Ctrl and Diab+2, N=6) and 8 months (FIGS. 5C-5D) of diabetes as revealed by ELISA analysis (P<0.05 vs. Ctrl and Diab+2, N=6).

FIGS. 6A-6D show representative Western blot and bar graph of the ratio of phosphorylated insulin receptor beta to total insulin receptor beta in the rat retina at 2 months (FIG. 6A, P<0.05 vs. Ctrl and Diab+2, N=5) and 8 months (FIG. 6B, P<0.05 vs. Ctrl and Diab+2, N=5). The overall ratio is substantially reduced at 8 months of treatment or control aging. Representative Western blot and bar graph of the ratio of phosphorylated Akt to total Akt in the rat retina at 2 months (FIG. 6C, P<0.05 vs. Ctrl and Diab+2, N=5) and 8 months (FIG. 6D, P<0.05 vs. Ctrl and Diab+2, N=5) are shown. The overall ratio is substantially reduced at 8 months of treatment or control aging.

FIGS. 6E-6H shows the effect of Compound 2 on the ratio of phospho-AKT to total AKT. FIGS. 6E-6F illustrate Akt phosphorylation in whole retinal lysates at 2 mo diabetes (left) and 8 months of diabetes (right). Treatment was initiated at the time of initial glucose measurement >250 mg/dl. FIGS. 6G-6H show that some animals were made diabetic with no intervention for 6 months. At 6 months, a subset of the diabetic animals were initiated on 1 mM topical Compound 2. At 8 months of diabetes (2 months, Compound 2) or 12 months of diabetic and 6 mo Compound 2, phosphorylation of Akt was measured in control, diabetic, and diabetic+Compound 2 treated rats.

FIGS. 7A-7D show ELISA analyses of cleaved caspase at 2 months (FIGS. 7A-7B) and 8 months (FIGS. 7C-7D). It is clear that apoptosis is increased in the retina in all groups at 8 months. *P<0.05 vs. Ctrl and Diab+49b; N=4 in each group at each age.

FIG. 8 shows the chemical structure of isoproterenol 1 (4-[1-hydroxy-2-(isopropylamino)ethyl]benzene-1,2-diol, Compound 2 (R)-4-[1-hydroxy-2-[3,4,5-trimethoxy-phenyl)-ethylamino]-ethyl)-benzene-1,2-diol hydrochloride and Compound 4 (R)-5-(1-hydroxy-2-[2-(3,4,5-trimethoxy-phenyl)-ethylamino]-ethyl)-benzene-1,3-diol hydrochloride.

FIGS. 9A-9B show that treatment of Müller cells cultured in high glucose with 10, 50 and 100 nM Compound 2. Treatment with Compound 2 significantly reduced levels of cleaved caspase 3 (FIG. 9A, P<0.05 vs. NT-HG, N=4) and TNFalpha at 50 nM as compared to treatment with isoproterenol, which required 10 uM for the same response (FIG. 9B, P<0.05 vs. NT-HG, N=4).

FIGS. 10A-10B show treatment of REC cells with 10, 50 and 100 nM Compound 2. Treatment with Compound 2 significantly reduced levels of cleaved caspase 3 (FIG. 10A, P<0.05 vs. NT-HG, N=4) and TNFalpha (FIG. 10B, P<0.05 vs. NT-HG, N=4) after 30 and 60 minutes as compared to treatment with isoproterenol.

FIGS. 11A-11B shows the effect in type I diabetic rats treated daily with 1 mM Compound 2.

FIGS. 12A-12B show that treatment of Müller cells with 50 nM Compound 3 reduced the cleavage of caspase 3 (FIG. 12A) vs. non-treated cells at 1 hour and significantly reduced TNFalpha within 1 hour compared to non-treated cells (FIG. 12B).

FIGS. 13A-13B show that treatment of REC cells with 50 nM Compound 3 reduced the cleavage of caspase 3 (FIG. 13A) and TNFalpha (FIG. 13B) vs. non-treated cells at 1 hour.

FIG. 14A-14B show the effects the R-isomer of Compound 2 (50 nM), the S-isomer of Compound 2 (50 nM) and racemic Compound 2 at either 1 hour (FIG. 14A) or 24 hours (FIG. 14B) of treatment on TNFalpha concentration in Muller and retinal endothelial cells.

FIGS. 15A-15B show the effects the R-isomer of Compound 2 (50 nM), the S-isomer of Compound 2 (50 nM) and racemic Compound 2 at either 1 hour (FIG. 15A) or 24 hours (FIG. 15B) of treatment on cleaved caspase 3 concentration in Muller and retinal endothelial cells.

FIG. 16 shows the levels of Compound 2 in the plasma of rats treated with 1 mg/kg intravenously. Compound 2 was not detected in the plasma of animal treated topically (N=5).

FIGS. 17A-17B are line graphs of Compound 2 in the plasma of rats treated intravenously (FIG. 17A) or topically (FIG. 17B) with 10 mg/kg. N=5 for both treatments. After 1 hour, numbers were below the 2.5 ng/ml limit of detection.

FIG. 18 shows levels of Compound 2 in the vitreous humor of rats treated with 10 mg/kg topical Compound 2. Data is mean±SD. N=5.

FIGS. 19A-19B illustrate the effect of Compound 2 on angiogenesis in proliferative diabetic retinopathy in hypoxic (FIG. 19A) and treated (FIG. 19B) mice. Treated mice received 1 mM Compound 2 as eye drops 1×/day for 3 days.

### DETAILED DESCRIPTION OF THE INVENTION

As used herein, the term “a” or “an”, when used in conjunction with the term “comprising” in the claims and/or the specification, may refer to “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “or” in the claims refers to “and/or” unless explicitly indicated to refer to alternatives only or the alternatives are mutually exclusive, although the disclosure supports a definition that refers to only alternatives and “and/or.”

As used herein, the term “contacting” refers to any suitable method of bringing one or more of the beta-adrenergic receptor agonists described herein or other inhibitory or stimulatory agent that improves retinal cell or retinal vascular tissue function and/or structure into contact with retinal cells, or a tissue comprising the same, associated with a diabetic condition, such as diabetic retinopathy or preproliferative retinopathy. In vitro or ex vivo this is achieved by exposing the retinal cells or tissue to the beta-adrenergic receptor agonists in a suitable medium. For in vivo applications, any known method of administration is suitable as described herein.

As used herein, the terms “effective amount” or “pharmacologically effective amount” are interchangeable and refer to an amount that results in a delay or prevention of onset of the diabetic-associated retinopathic condition or results in an improvement or remediation of the symptoms of the same. Those of skill in the art understand that the effective amount may improve the patient's or subject's condition, but may not be a complete cure of the condition. As used herein, the term “subject” refers to any target of the treatment.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

In one embodiment of the present invention there is provided a method for improving function in a retinal cell associated with a diabetic condition, comprising contacting the cell with a beta-adrenergic receptor agonist, where the beta-adrenergic receptor agonist increases insulin signaling and decreases TNFα-induced apoptosis, thereby improving the function in the retinal cell. In this embodiment the beta-adrenergic receptor agonist may have the chemical structural formula:

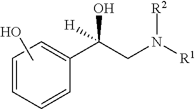

where R¹ is (CH₂)ₙ(CH₃)₂ or is

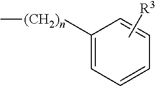

where n is 1 to 4; R² is H or H.HX, where X is a halide; and R³ is O(CH₂)ₘCH₃ at one or more of C2-C6, where m is 0 to 4.

In one aspect of this embodiment R¹ may be (CH₂)ₙ(CH₃)₂ and R² may be H. In another aspect R¹ may be (CH₂)₂-phenyl, R² may be H or H.HCl and R³ may be O(CH₂)ₘCH₃ at C3, C4 and C5. In this other aspect the beta-adrenergic receptor agonist may be (R)-4-[1-hydroxy-2-[3,4,5-trimethoxy-phenyl)-ethylamino]-ethyl)-benzene-1,2-diol, (R)-4-[1-hydroxy-2-[3,4,5-trimethoxy-phenyl)-ethylamino]-ethyl)-benzene-1,2-diol hydrochloride, (R)-5-(1-hydroxy-2-[2-(3,4,5-trimethosy-phenyl)-ethylamino]-ethyl)-benzene-1,3-diol, or (R)-5-(1-hydroxy-2-[2-(3,4,5-trimethoxy-phenyl)-ethylamino]-ethyl)-benzene-1,3-diol hydrochloride. In all aspects and embodiments the retinal cell may be contacted in vitro or in vivo. Representative diabetic conditions include but are not limited to diabetic retinopathy, preproliferative diabetic retinopathy, proliferative diabetic retinopathy or other hyperglycemic conditions.

In another embodiment of the present invention there is provided a method for treating a diabetic retinopathic condition in a subject, comprising administering one or more times a pharmacologically effect amount of one or more beta-adrenergic receptor agonists to the subject, where the agonist improves retinal cell function, thereby treating the diabetic retinopathy. Further to this embodiment the method comprises administering one or more other diabetic or retinopathic drugs to the subject. In this further embodiment the other drugs may be administered concurrently or sequentially with the beta-adrenergic receptor agonist(s).

In both embodiments the beta-adrenergic receptor agonists may be as described supra. Also, in both embodiments the diabetic retinopathic condition may be preproliferative retinopathy. In addition, the beta-adrenergic receptor agonists may comprise a pharmaceutical composition with a pharmaceutically acceptable carrier, which is suitable for topical, subconjunctival or intravenous administration.

In another embodiment of the present invention there is provided a beta adrenergic receptor agonist having the chemical structural formula:

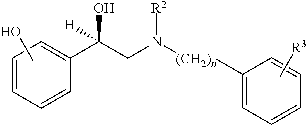

where n is 1 to 4 and R² is O(CH₂)ₘCH₃ at one or more of C2-C6, where m is 0 to 4. In an aspect of this embodiment, n is 2, R² is H or H.HCl and R³ is OCH₃ at C3, C4 and C5. The beta-adrenergic compound may be in the R-isomeric form: Particularly, the beta-adrenergic receptor agonist may be (R)-4-β-hydroxy-2-[3,4,5-trimethoxy-phenyl)-ethylamino]-ethyl)-benzene-1,2-diol, (R)-4-β-hydroxy-2-[3,4,5-trimethoxy-phenyl)-ethylamino]-ethyl)-benzene-1,2-diol hydrochloride, (R)-5-(1-hydroxy-2-[(3,4,5-trimethosy-phenyl)-ethylamino]-ethyl)-benzene-1,3-diol, or (R)-5-(1-hydroxy-2-[(3,4,5-trimethoxy-phenyl)-ethylamino]-ethyl)-benzene-1,3-diol hydrochloride.

In a related embodiment, the present invention provides a pharmaceutical composition comprising the beta-adrenergic receptor agonist as described supra and a pharmaceutically acceptable carrier.

There is an unexpected overlap between insulin receptor and β-adrenergic receptor signaling. Importantly, increased beta-adrenergic receptor signaling may compensate for loss of insulin signaling in diabetes, as demonstrated by a decrease in apoptotic cell death in diabetic rats after treatment with beta-adrenergic receptor agonists. The cellular mechanisms involved may include a direct compensatory effect of beta-adrenergic receptor signaling on cell death or alternatively, an inhibition prevention of insulin receptors through pathways involving inflammatory mediators such as TNFalpha. It may also involve an upregulation of IGFBP-3 to inhibit retinal endothelial cell death.

The present invention provides derivative and analog compounds of Compound 2. Both Compound 2 and the derivative/analog compounds are beta-adrenergic receptor agonists. These compounds have catecholaminergic properties and also activate both beta-1- and beta-2-adrenergic receptors. These compounds are compared with isoproterenol in some embodiments. It is demonstrated that while both isoproteronol and the compounds of the present invention have beta-adrenergic receptor activities, although isoproterenol is a non-selective agonist, the beta-adrenergic receptor agonists of the present invention have more potent and specific effects than isoproterenol.

The beta-adrenergic receptor agonists of the present invention provided herein may be synthesized by known and standard chemical synthetic methods. Generally, these beta-adrenergic receptor agonists, including the known isoproterenol, may have the chemical structure:

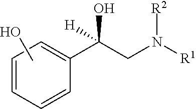

The R¹ substituent may comprise the moiety (CH₂)ₙ(CH₃)₂, where n is 1 to 4, for example, the isopropyl moiety CH₂(CH₃)₂ as in isoproterenol 1 or may comprise a substituted phenyl moiety:

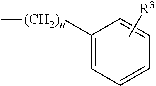

where n is 1 to 4. R² is either hydrogen or a pharmacologically acceptable salt or hydrate moiety, such as H.HX, where X is a halide, for example, but not limited to chloride. R³ is substituted at one or more of the C2-C6 phenyl carbons where R³ is independently —O(CH₂)ₘCH₃ and m is 0 to 4.

Generally, the novel beta-adrenergic receptor agonists provided herein include a benzene diol moiety. For example, the beta-adrenergic receptor agonist may have the chemical structure:

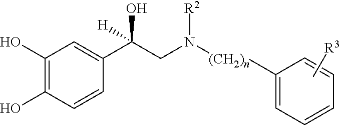

Preferred beta-adrenergic receptor agonists with a benzene 1,2-diol moiety are 4-[1-hydroxy-2-(1-ethylamino-3-,4-,5-trimethoxyphenyl)ethyl]benzene-1,2-diol hydrochloride (Compound 2) or the R-isomer thereof and has the chemical structure:

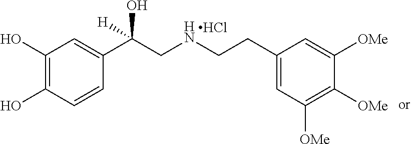

4-[1-hydroxy-2-(1-ethylamino-3-,4-,5-trimethoxyphenyl)ethyl]benzene-1,2-diol (Compound 3) or the R-isomer thereof with the chemical structure:

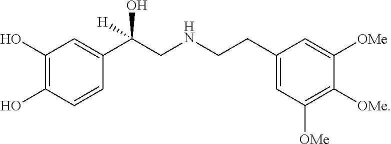

In addition, the beta-adrenergic receptor agonist may have the chemical structure:

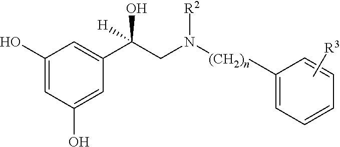

More preferable beta-adrenergic receptor agonists with a benzene 1,3-diol moiety are 5-(1-hydroxy-2-[2-(3,4,5-trimethosy-phenyl)-ethylamino]-ethyl)-benzene-1,3-diol hydrochloride (Compound 4) or the R-isomer thereof with the chemical structure:

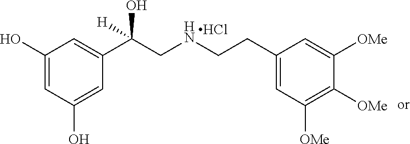

5-(1-hydroxy-2-[2-(3,4,5-trimethoxy-phenyl)-ethylamino]-ethyl)-benzene-1,3-diol (Compound 5) or the R-isomer thereof with the chemical structure:

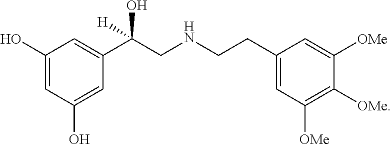

It is determined that the potential mechanism of action of Compound 2 and other beta-adrenergic receptor agonists described herein is through the reduction of TNFalpha and increased insulin signaling for Müller cells and through increased IGFBP-3 levels in retinal endothelial cells. It is contemplated that these actions may represent biomarkers for human diabetic retinopathy. The present invention demonstrates that beta-adrenergic receptor agonists prevent damage caused by diabetes or hyperglycemic conditions that damage multiple retinal cell types. A critical feature of treatment with the beta-adrenergic receptor agonists presented herein is the selective specificity, i.e., while they do reduce retinal damage, they do not reduce blood pressure, alter intraocular pressure, and are significantly more efficacious than current angiotensin converting enzyme agents.

Thus, the present invention also provides methods of decreasing or preventing diabetic-associated retinal damage to retinal cell function and structure and to retinal tissue capillaries, such as by preventing and/or reversing diabetic retinopathy, for example, proliferative diabetic retinopathy, through compensation for or maintenance of insulin receptor signaling. These methods may be performed in vitro or in vivo. For example, contacting a retinal cell associated with a diabetic condition with a beta-adrenergic receptor agonist improves retinal function of the cell by inter alia increasing insulin signaling and decreasing TNFalpha-induced apoptosis.

Particularly, the in vivo treatment methods provided herein target the pre-proliferative phase of diabetic retinopathy when clinically observable symptoms are not evident and before cell death and resulting vision loss occurs. Treatment is effected via administration of one or more of the beta-adrenergic receptor agonists or pharmacologically effective and acceptable salts or hydrates thereof described herein. Pharmaceutical compositions comprising the beta-adrenergic receptor agonists and a pharmaceutically acceptable carrier as is known and standard in the art also may be administered. It is contemplated that one or more other diabetic or retinopathic drugs or therapeutic agents may be administered concurrently or sequentially with the beta-adrenergic receptor agonist(s).

Dosage formulations of the beta-adrenergic receptor agonist compounds or a pharmacologically acceptable salt or hydrate thereof may comprise conventional non-toxic, physiologically or pharmaceutically acceptable carriers or vehicles suitable for the method of administration. Methods of administration are known in the art, preferably, subconjunctival delivery and topical delivery, but may include intravenous delivery. These compounds or pharmaceutical compositions thereof may be administered independently one or more times to achieve, maintain or improve upon a pharmacologic or therapeutic effect derived from these compounds or other anti-diabetic drugs or agents. It is well within the skill of an artisan to determine dosage or whether a suitable dosage comprises a single administered dose or multiple administered doses. An appropriate dosage depends on the subject's health, the progression or stage of the diabetes and/or retinopathy, the route of administration and the formulation used.

The following examples are given for the purpose of illustrating various embodiments of the invention and are not meant to limit the present invention in any fashion.

### Example 1

### Methods

### Animal Preparation

Male Lewis rats purchased from Charles River were used. Diabetic rats received a single injection of 60 mg/kg streptozotocin (Fisher, Pittsburgh, Pa.). The control rats received an injection of citrate buffer. All rats were weighed weekly and only rats with non-fasting blood glucose levels >250 mg/dl were considered diabetic. The designation of diabetic was made at the beginning of the experiments. Glucose was measured bimonthly. No insulin was administered to the rats at any time.

To determine whether topical application of Compound 2 could reach the retina and elicit response, dose and time course studies were performed. For those studies, animals were made diabetic for 2 months using STZ. For 4 days, rats received a variety of doses of either Compound 2 (1 mM to 20 mM) or PBS once or twice each day. After the 4 days of eye drop treatment to both eyes, the diabetic and control animals were euthanized. Since beta-adrenergic receptor stimulation should increase cyclic adenosine monophosphase production and activate protein kinase A, retinal lysates were processed for a PKA ELISA (MesaCup PKA ELISA, Upstate, Temecula, Calif.). After determining the optimal dose and time course for eye drop treatment for Compound 2, three groups of rats were used for this part of the study (control, diabetic (Diabetic), and diabetic+eye drop (Diab+2). Within 1 week of STZ-injection, 12 rats were put onto eye drop therapy. Rats on the eye drop therapy received a daily application of 4 drops of 1 mM Compound 2 to each eye. To verify that insulin levels were lost in the diabetic animals, insulin ELISA (Rat/Mouse Insulin ELISA kit, Linco, St. Charles, Mo.) was done on blood sampled from all rats at 2 months of age. Rats were sacrificed at 2 or 8 months of age. At 2 or 8 months, retinas were assessed for diabetes-induced degeneration of retinal cell numbers and retinal thickness (2 months) and for degenerate capillaries (8 months). TNF activity, cleaved caspase 3 and phosphorylation of insulin receptor beta and Akt were assessed at both time points.

### Electroretinograms

Each month of the experiment, electroretinogram (ERG) analyses were performed on rats from the three groups. Electroretinogram analyses were done to evaluate changes in electrical activity of the retina and as a measure of drug effectiveness. For the electroretinogram analyses, rats were dark adapted overnight. The following morning, the rats were anesthetized using an intraperitoneal injection of a ketamine (0.6 ml/kg body weight) and xylazine (0.375 ml/kg body weight) cocktail. The pupil of each eye was fully dilated using a 1% tropicamide solution (Alcon). To protect the eye and assist in maintaining a good electrical connection, a drop of methylcellulose solution was added to each eye (Celluvisc; Allergan, Irvine, Calif.). Body temperature was maintained at 37° C. with a water-based heating pad. The electroretinogram responses were recorded from both eyes simultaneously using platinum wire corneal electrodes, a forehead reference electrode, and ground electrode in the tail. The electroretinogram stimuli were delivered via the Diagnosys LLC system. All animals tested recovered from anesthesia after the electroretinogram recording sessions. No animals with gross cataract were used for electroretinogram analyses.

Electroretinogram responses were recorded in response to brief (4 ms) white LED and then from the Xenon arc lamp delivered at 2.1-second intervals for dim stimuli and 35 second frames for brighter stimuli. The range of stimulus intensities extended from −4.0 to 1.0 log cd*s/m2 for analysis of the b-wave amplitudes. Electroretinogram waveforms were recorded with a bandwidth of 0.3-500 Hz and sampled at 2 kHz by a digital acquisition system (Diagnosys) and were analyzed using MatLab (The MathWorks, Natick, Mass.). Plots of intensity-response functions for the a-wave and b-waves were fit to a hyperbolic (Naka-Rushton) function of the form R(I)/Rmax=Ik/Ik+Kn where R was the response amplitude at flash intensity I, Rmax was the amplitude of the maximal response that can be elicited; and K was the intensity that evokes a half-maximal response.

For assessment of the oscillatory potentials, stimuli were administered at 3 log (cd*s/m2). Data analysis for the oscillatory potentials was obtained using MatLab software with a digital band-pass filter set for 60-300 Hz and the peak wavelets of the 4 wavelets were measured from trough to peak. (3-4). Statistics were done on the mean SD amplitudes of the a- and b-wave of each treatment group at 2-, 6- and 8-months.

### Preparation of Trypsin-Digested Retinal Vasculature

For the acellular capillary counts, retinas from an eye of control, diabetic, and diabetic+2 were used. Eyes were enucleated and placed into 10% buffered formalin for 5 days. The retina was dissected in 3% crude trypsin solution (Difco Bacto Trypsin 250, Detroit, Mich.) containing 0.2 M sodium fluoride at 37 C for 2 hours (5). The neural retina was gently brushed away and the remaining retinal vascular tree was dried onto a glass slide.

### Quantitation of Acellular Capillaries

Once the isolated retinal vascular tree was dried onto the glass slide, the slide was stained with hematoxylin-periodic acid-Schiff. Degenerate (acellular) capillaries were counted in mid-retina in six to seven fields evenly spaced around the retina. Degenerate capillaries were identified as capillary-sized tubes having no nuclei anywhere along their length. Degenerate capillaries were counted only if their average diameter was at least 20% of that found in surrounding healthy capillaries (6-7).

Assessment of retinal thinning and loss of cells in ganglion cell layer Formalin-fixed paraffin sections were stained with toluidine blue for light microscopy and morphometry of retinal thickness (8). Pictures were taken at four locations in the retina (both sides of the optic nerve and mid-retina) at 400×. The nuclei in the retinal ganglion cell layer (GCL) were counted in a 100 m section of each picture, and the thickness of the inner retina (from the top of the inner nuclear layer to the inner limiting membrane) was assessed using a Retiga camera attached to a Nikon Biophot light microscope with Qcapture software (Qlmaging, Burbay, BC, Canada). Retinal thickness and number of cells in the ganglion cell layer were measured using OpenLab software (Improvision, Lexington, Mass.).

### Protein Analyses

The other eye from each animal was used for protein analyses for inflammatory markers and insulin receptor signaling. Western blotting was done as described (9). Antibodies used were total insulin receptor beta (1:500, Cellular Signaling, Danvers, Mass.), phosphorylated insulin receptor beta (Tyr 1150/1151, 1:500, Cellular Signaling, Danvers, Mass.), total Akt (1:500, Cellular Signaling, Danvers, Mass.), and phosphorylated Akt (Ser 473, 1:500, Cellular Signaling, Danvers, Mass.). For analyses of the data, mean densitometry values were obtained using the Kodak 2.0 software. The ratio of phosphorylated protein was compared to levels of total protein.

### ELISA Analyses

To determine the TNF concentration (Pierce, Rockford Ill.) and levels of cleaved caspase 3 (Cellular Signaling, Danvers, Mass.), ELISA analyses were done according to manufacturers instructions, except that equal protein was loaded into the cleaved caspase 3 ELISA so the optical density numbers can be used.

### Cells

Human retinal endothelial cells (HREC) were purchased from Cell Systems (Kirkland, Wash.) and grown in either basal (5 mM glucose) or growth (25 mM glucose) media. Both media is supplemented with 10% FBS and antibiotics. The day prior to experiments, cells are serum-starved for 18-24 hours. Rat Müller cells (rMC-1) were grown in DMEM medium with 5 mM glucose or 25 mM glucose. Media was supplemented with 10% FBS and antibiotics. Cells were serum starved prior to all experiments for 18-24 hours.

### Preparation of Membranes and Radioligand Binding Assays

REC are cultured on 10 cm-culture plates, washed twice with 10 ml ice-cold PBS, then scraped from the plates and pelleted by centrifugation at 2,000 gav for 10 min. The cell pellets are suspended in 10 ml of hypotonic buffer composed of 20 mM HEPES, pH 7.4, 2 mM MgCl2, 1 mM EDTA and 1 mM 2-mercaptoethanol supplemented with 10 μg/ml leupeptin and 10 μg/mlaprotinin (with or without 1 mM phenylmethyl sulfonyl fluoride) for 10 min on ice. The cells are lysed by 30 up-and-down strokes in a glass-glass homogenizer then centrifuged at 2,500 gav for 5 min. The supernatant is re-centrifuged at 15,000 gav for 20 min to pellet the membranes. Binding of the highly selective ligand [125I] iodocyanopindolol (ICYP) to 0.5 μg of membranes is measured in 50 mM Tris-HCl, pH 7.4 plus 10 mM MgCl2 binding buffer containing 0.1 mM ascorbic acid for 2 h at 25° C. For saturation binding experiments, ICYP concentrations ranging between 5 and 300 pM are used to calculate the KD and the Bmax for ICYP binding by parametric fitting of the data using the Prism 4 software.

### Statistics

Statistics were done to compare the control, diabetic, and diabetic-Ftreatment (Diab+Compound 2) using a Kruskal-Wallis analysis, with Dunn's test for post-hoc analyses. P<0.05 was taken as significant.

### Example 2

### In Vivo Effects of Compound 2

### Compound 2 Did not Affect Body Weight or Glucose Levels

The daily administration of 1 mM Compound 2 did not affect body weight or blood glucose levels (Table 1). Plasma concentration of Compound 2 decreased from about 100 ng/ml to about 6 ng/ml over 45 minutes. Body weight and blood glucose levels showed little variation between the 2 and 8-month time points. There was also no observed effect on blood pressure or intraocular pressure following Compound 2 treatment (Table 1). Normal insulin levels were measured in the control retina, while the diabetic and diabetic+Compound 2 animals had little to no insulin.

TABLE 1

| | | Body wt | | BP | BP | |
|---|---|---|---|---|---|---|
| | N | (g) | Glucose | Systolic | Diastolic | IOP |
| Ctrl | 6 | 506.7 ± 37.5 | 132.7 ± 7.3 | 100.0 ± 10.9 | 77.0 ± 9.4 | 8.92 ± 1.52 |
| Diabetic | 8 | 273.1 ± 40.5** | 724.6 ± 39.2*** | 115.9 ± 14.1 | 87.8 ± 13.7 | 7.19 ± 1.05* |
| Diab + 2 | 6 | 248.7 ± 8.3** | 695.8 ± 62.2*** | 104.8 ± 13.7 | 9½ ± 14.1 | 8.08 ± 1.17 |

### Compound 2 Increased PKA Activity

To determine the optimal dose and time frame for drug administration, rats treated with STZ for 2 months were used and treated for 4 days with varying doses of 4 drops of Compound 2 to each eye. In the initial study, the optimal dose range investigated was between 1 mM to 20 mM, given once per day. Measurement of PKA was used as a biomarker that Compound 2 was reaching the retina and eliciting a normal cellular response, since beta-adrenergic receptors normally activate PKA.

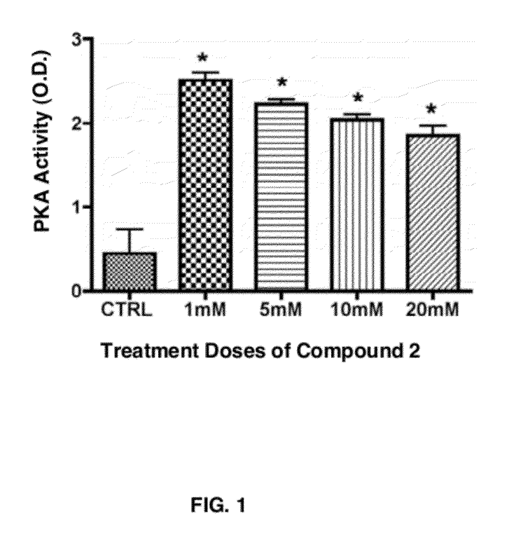

A topical dose of 1 mM given once daily showed the highest increase in PKA activity in comparison with the other administered doses (FIG. 1 P<0.05 vs. Ctrl, N=6). The 1 mM concentration treatment was used for all subsequent experiments.

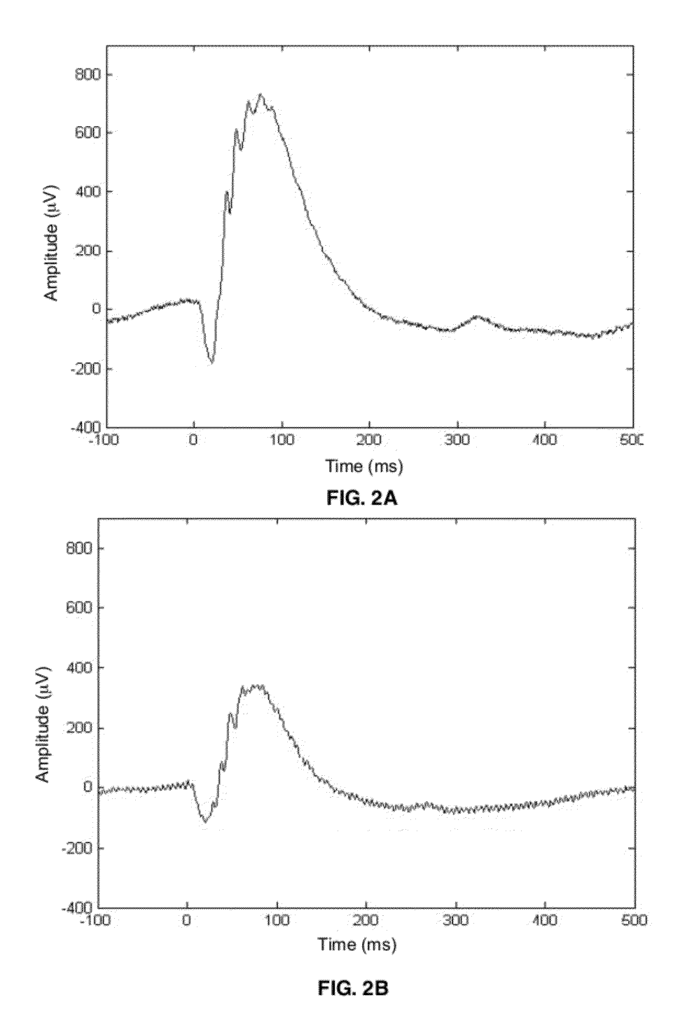

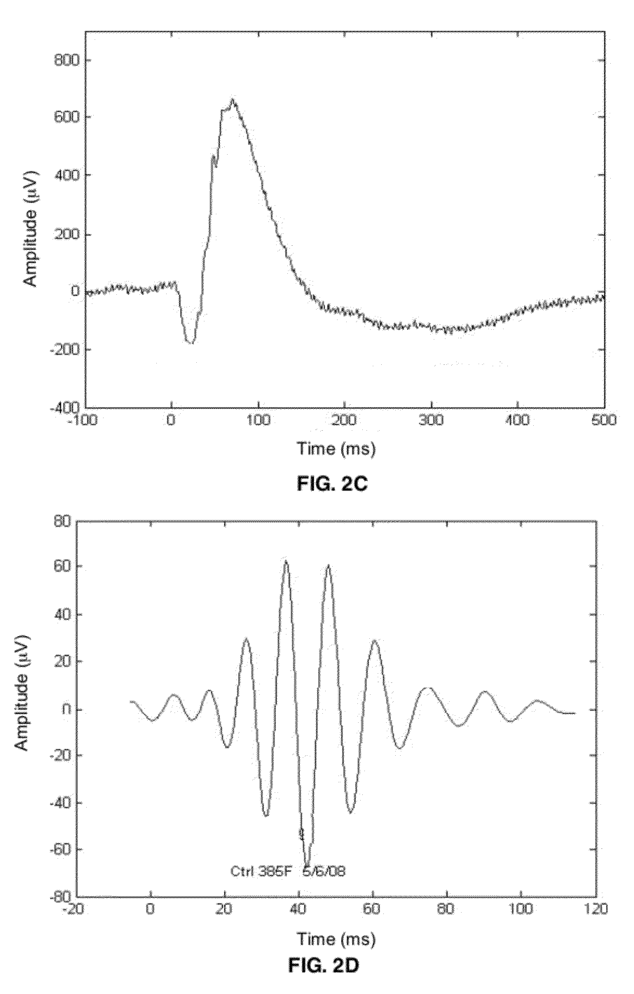

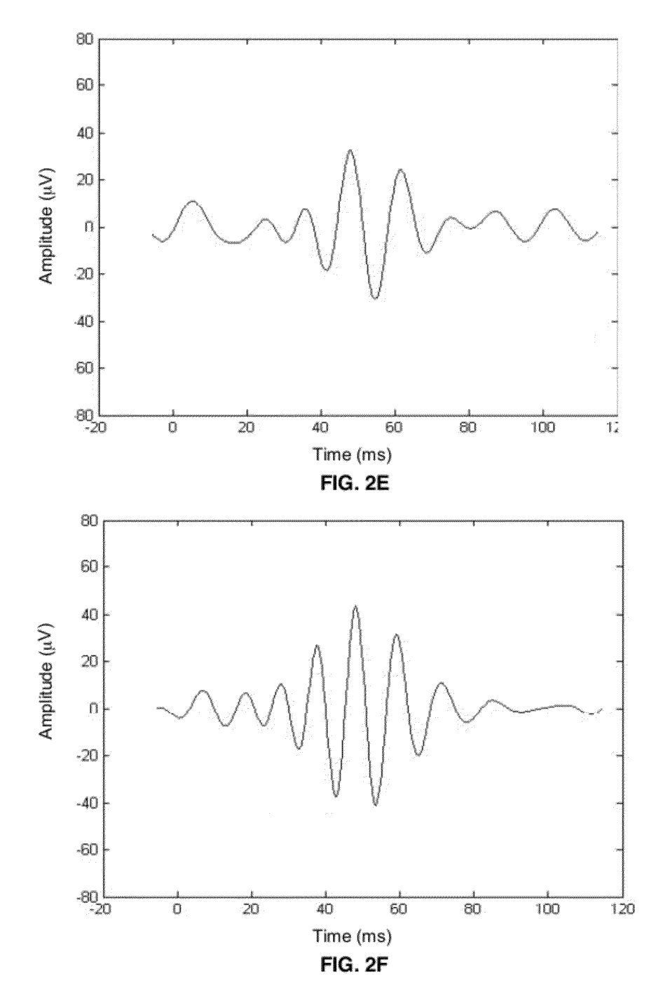

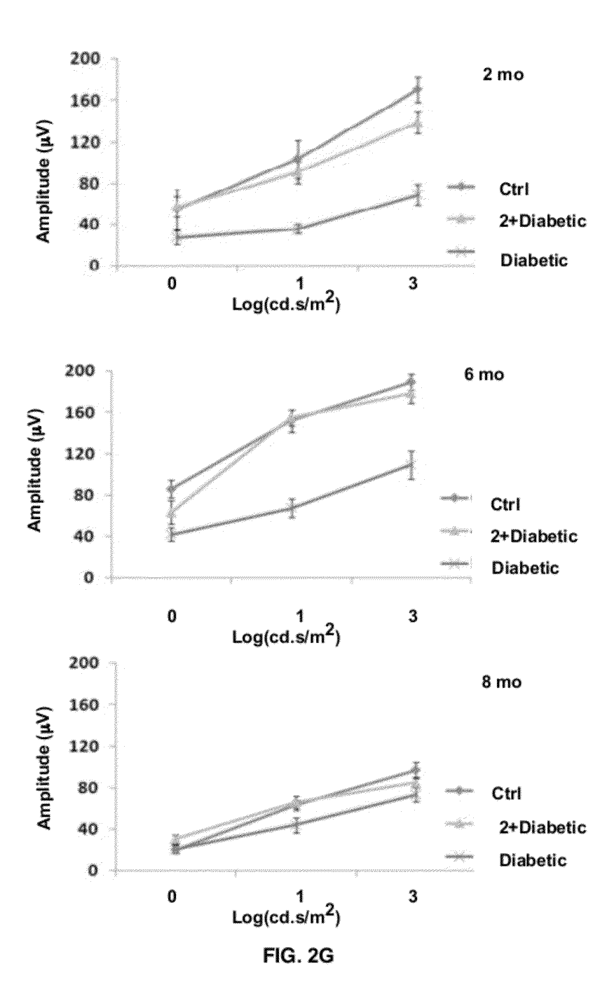

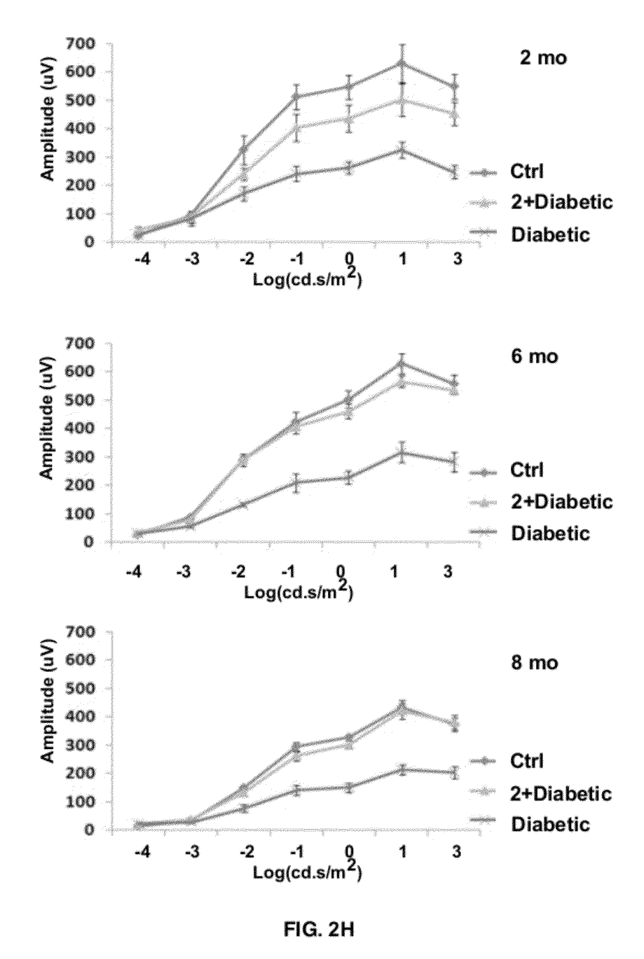

Compound 2 inhibited the loss of amplitude of B-wave and oscillatory potentials in the electroretinogram over 8 months (FIGS. 2A-2F). Electroretinogram analyses of visual function were done each month on the control, diabetic, and diabetic+eye drop treated animals. The amplitudes of the a-wave (FIG. 2G), b-wave (FIG. 2H) and oscillatory potentials (FIG. 2I) were substantially reduced in the diabetic animals within 2 months of diabetes, which was maintained over the 8-month period. Little difference was observed in ERG amplitudes between the control rats and those diabetic rats receiving Compound 2 treatment. These results suggest that the eye drop was effective at maintaining electrical activity of the retina in spite of diabetes in the rats.

Inner Retinal Thickness and Numbers of Cells in the Ganglion Cell Layer of the Central Retina were Maintained in Eye Drop Treated Diabetic Rats

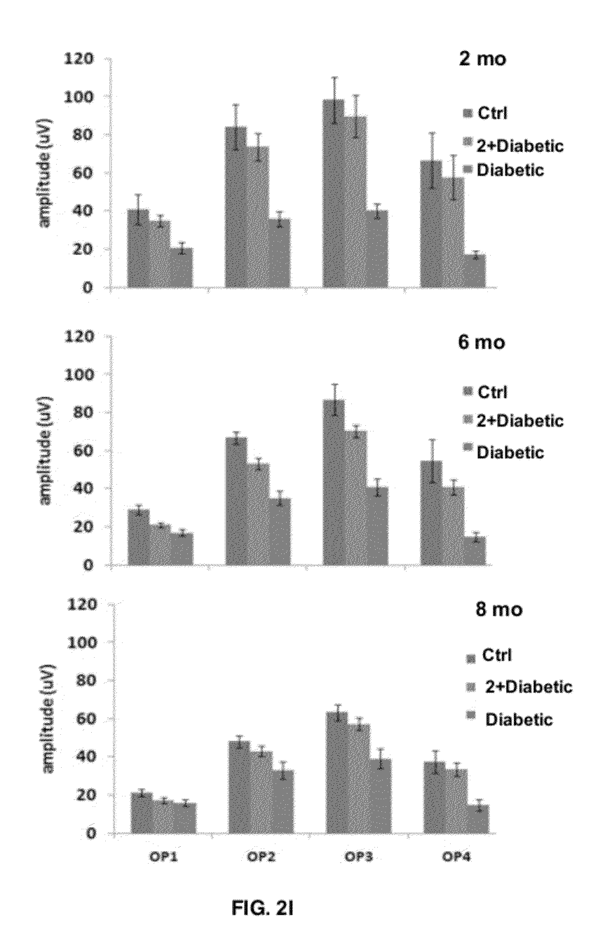

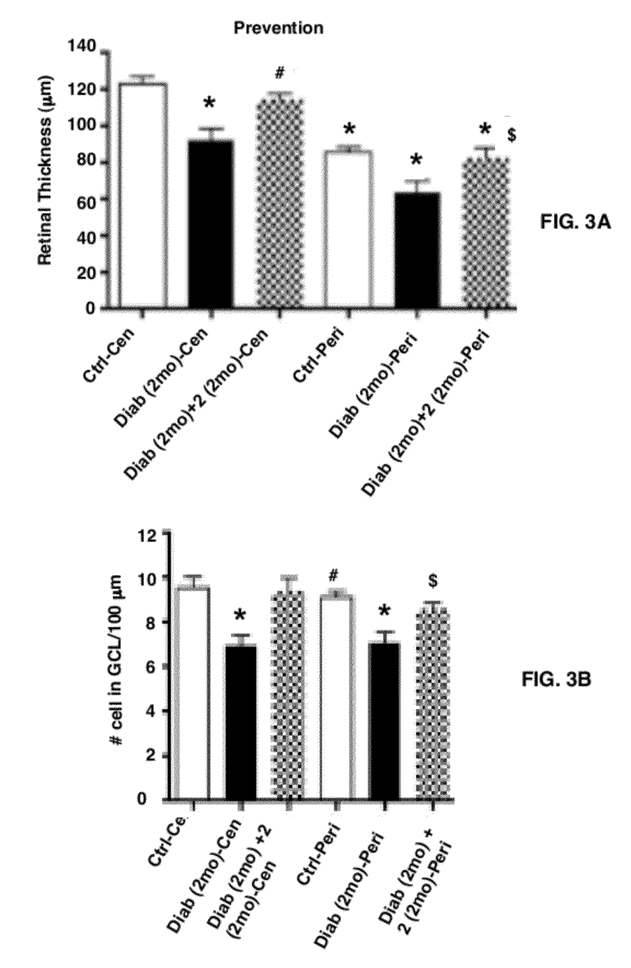

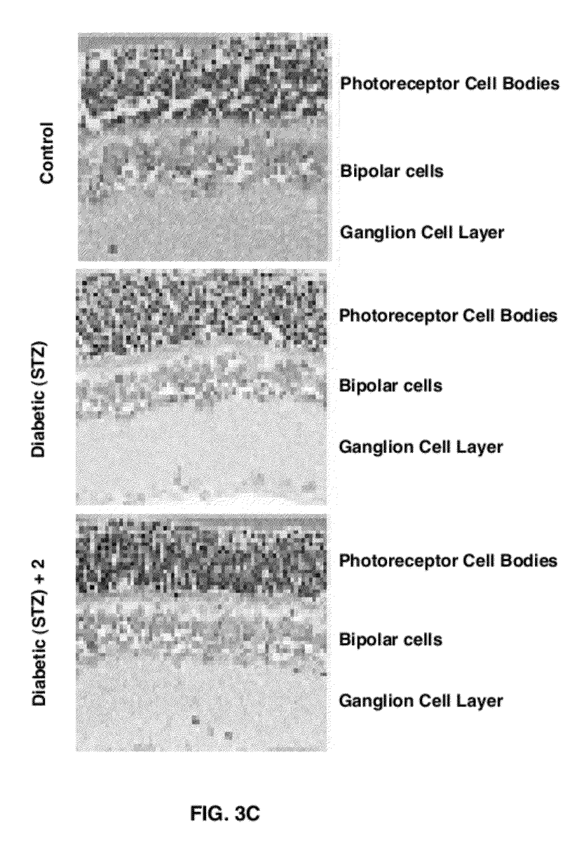

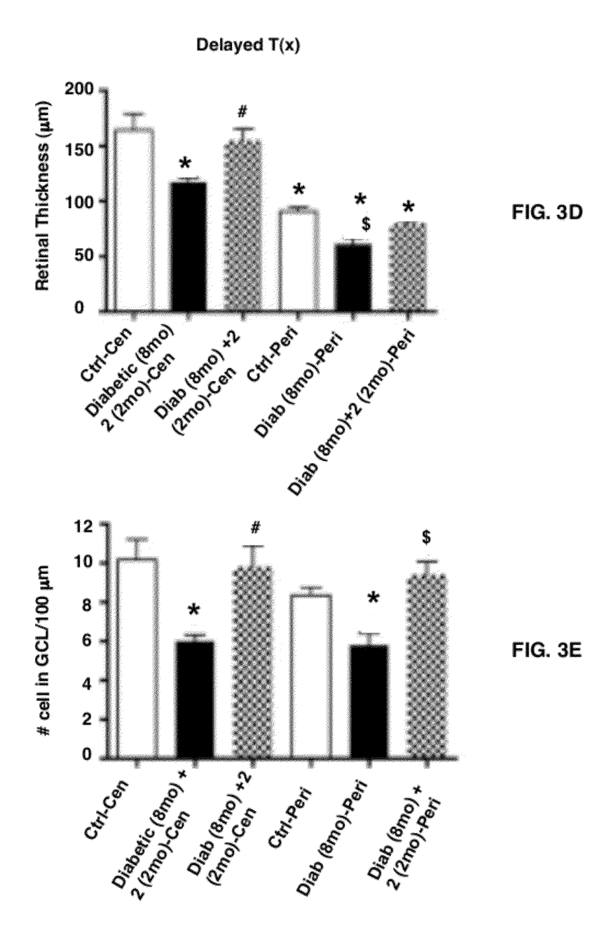

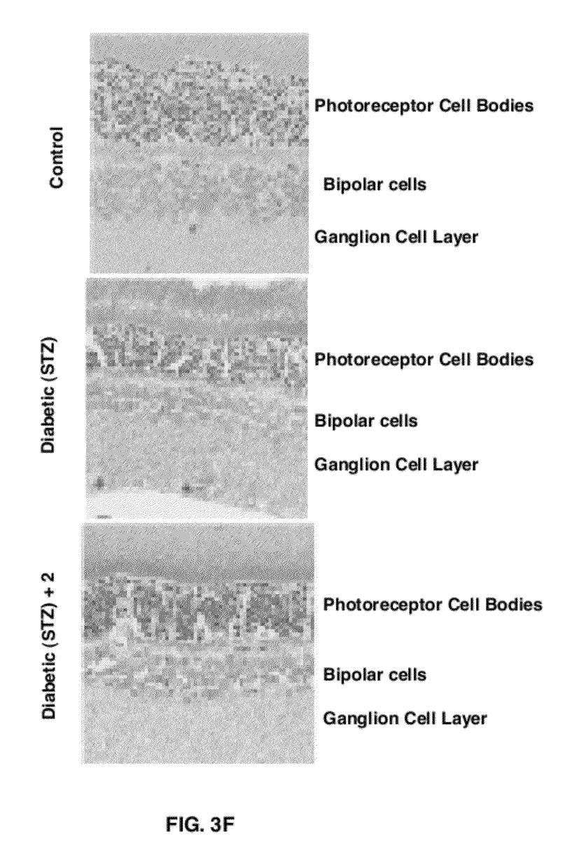

Retinal thickness near the optic nerve (central retina) was significantly reduced in the diabetic rats compared to control (FIGS. 3A-3C). This loss of inner retinal thickness was prevented in diabetic rats receiving eye drop treatment. Similarly, diabetic rats had fewer cells noted in the central retinal regions (FIGS. 3D-3F), which was prevented in the Compound 2-treated animals. It is likely that the reduced numbers of cells in the ganglion cell layer are both retinal ganglion cells and displaced amacrine cells. No changes in retinal thickness or cell numbers were noted in the peripheral retina, away from the optic nerve).

### Compound 2 Therapy Prevented the Degeneration of Retinal Capillaries

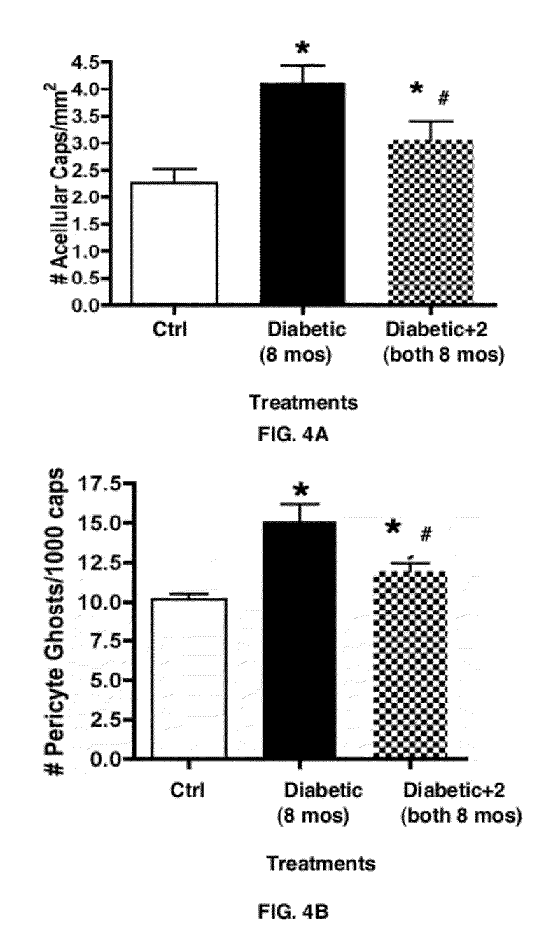

Numbers of degenerate capillaries are a key finding of vascular changes in the diabetic rat retina (6-7). Treatment with 1 mM Compound 2 significantly reduced numbers of degenerate capillaries in diabetic animals to levels similar to control animals after 8 months of diabetes (FIGS. 4A-4B, P<0.05 vs. ctrl and diab+2).

Tnfalpha Levels were Significantly Reduced in the Compound 2-Treated Animals

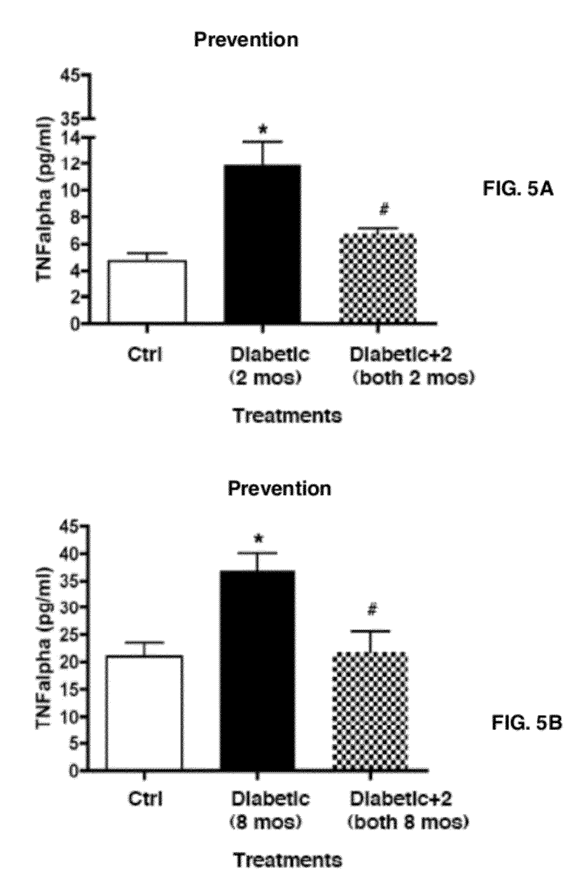

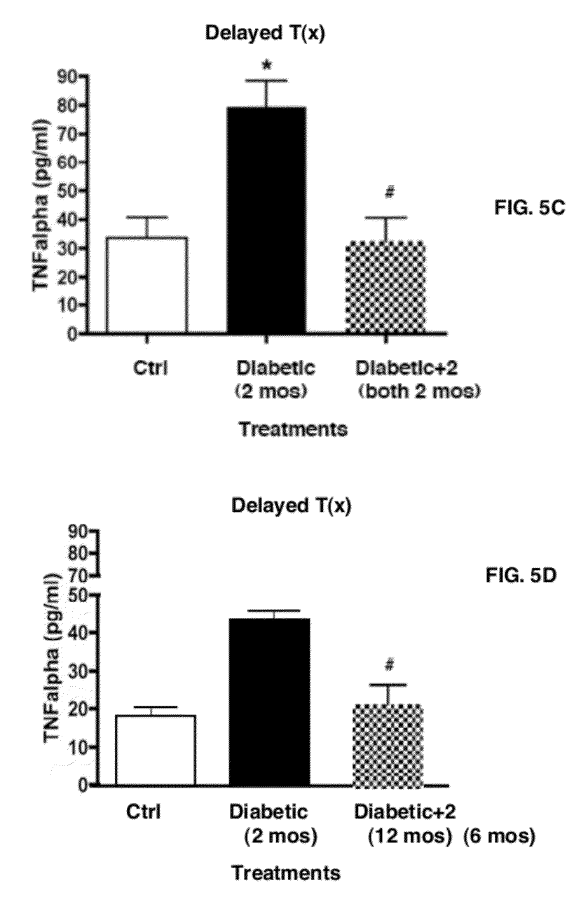

Because of a significant reduction in TNFa levels observed in vitro, levels were also assessed to ensure that Compound 2 was able to decrease inflammatory marker levels in diabetic rats. Data show that after 2 months of treatment protein levels of TNFalpha were significantly elevated in diabetic only rats. While the same levels in Compound 2 treated rats were similar to control levels (FIGS. 5A-5D, P<0.05, vs. Ctrl and Diab+2, N=6). Similarly, TNFalpha levels were significantly elevated in diabetic only rats while treated rats showed levels similar to Ctrl rats after 8 months of Compound 2 treatment (FIGS. 5C-5D, P<0.05 vs. Ctrl, N=6). These results suggest that beta-adrenergic receptor agonists can reduce inflammatory marker levels both in culture and in a physiologically relevant model.

### Compound 2 Inhibited Loss of Insulin Receptor Tyrosine Phosphorylation in Diabetic Rats

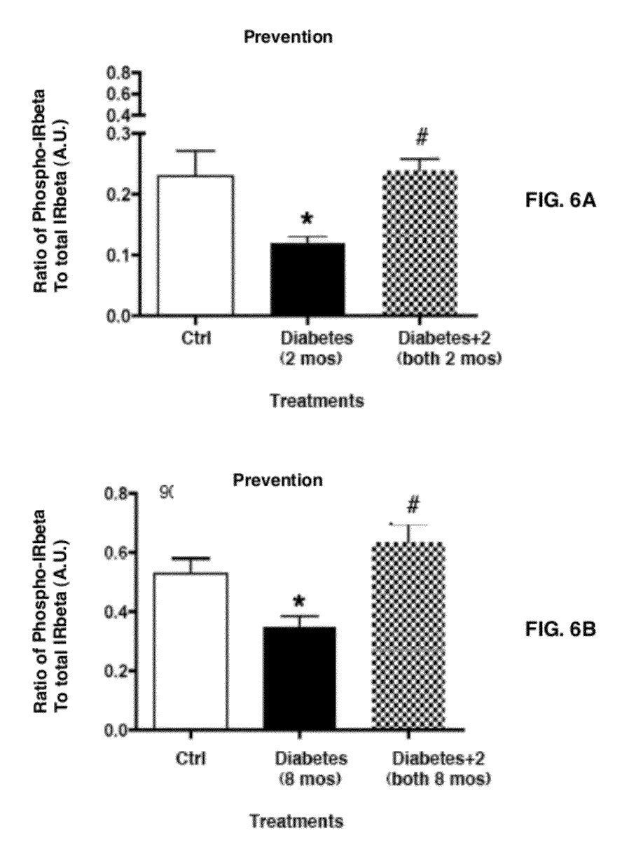

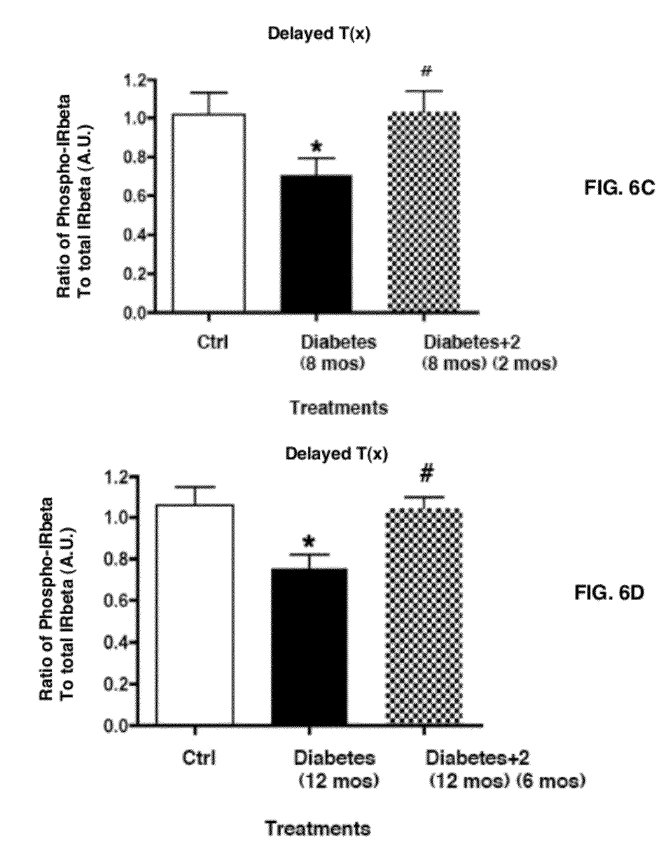

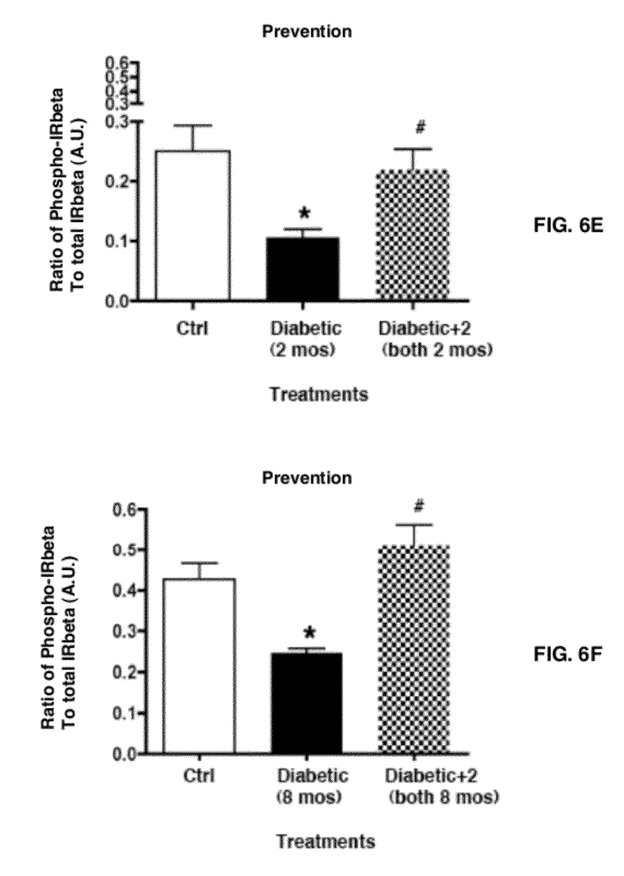

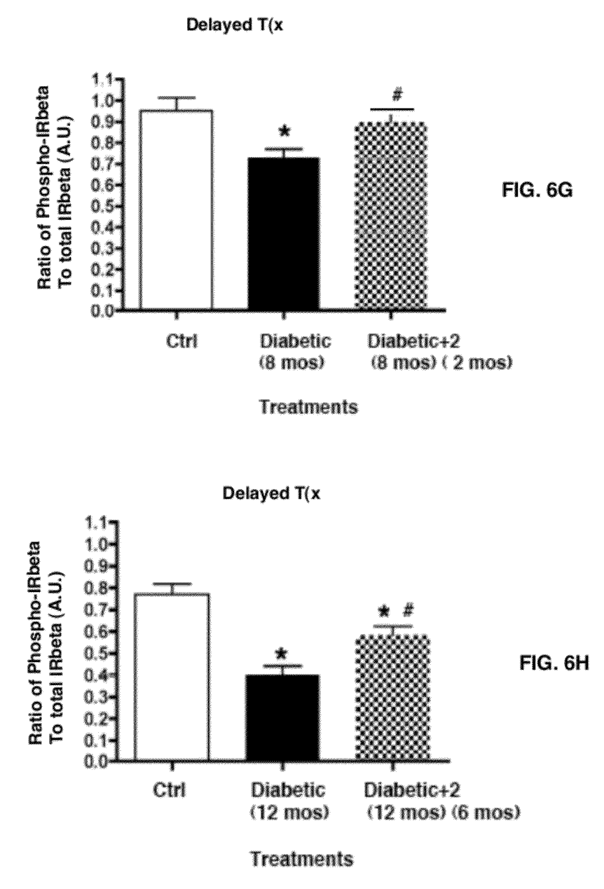

It has been reported that stimulation of the insulin receptor beta occurs primarily at the tyrosine 1150/1151 residues in the rats and that insulin receptor signaling occurs in the retina (2,10). Treatment with Compound 2 maintained insulin receptor beta tyrosine phosphorylation at levels similar to control animals after 2 months of diabetes (FIGS. 6A-6C, P<0.05 vs, control) as compared to diabetic rats only. Insulin receptor beta tyrosine phosphorylation was also maintained after 8 months of diabetes in rats treated with Component 2 (FIGS. 6D-6E, P<0.05 vs. control). Since Akt phosphorylation is indicative of cell survival, protein levels of both total Akt and phosphorylated Akt were analyzed in retinal lysates of rats from each treatment group. Daily treatment with 1 mM of Compound 2 maintained the ratio of phosphorylated Akt at levels similar to control values while diabetes significantly decreased Akt phosphorylation (FIG. 6G, P<0.05 vs. control). Protein levels were significantly decreased in diabetic only rats when compared to control rats after 8 months (FIG. 6H, P<0.05 vs. control and Diab+2, N=5). Similarly, FIGS. 6E-6H shows the effect of Compound 2 on the ratio of phospho-AKT to total AKT.

### Decreased Cleaved Caspase 3 Levels in Eye Drop Treated Animals

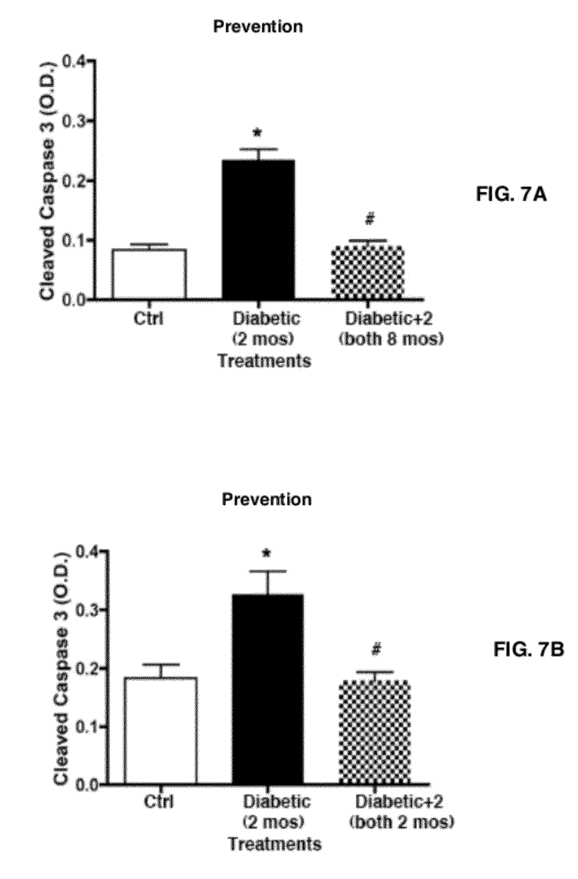

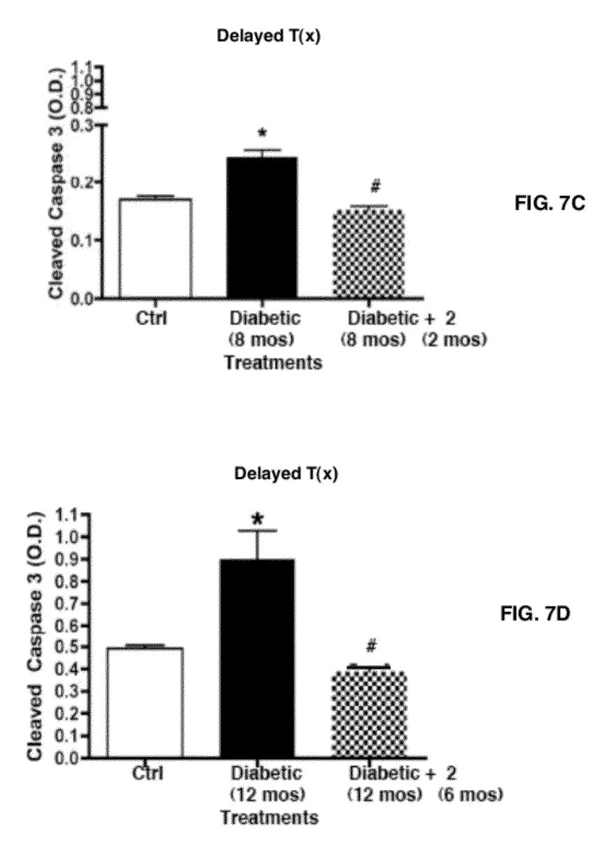

Since there was a reduction in diabetes-induced cell loss in the treated animals in the central retina, apoptosis is likely reduced following treatment. Akt phosphorylation was maintained due to eye drop treatment to diabetic rats, again suggesting that apoptosis would be reduced. Indeed, diabetes produced a significant increase in cleaved caspase 3 levels in retinal lysates (P<0.05 vs. control), which was reduced following treatment with the Compound 2 eye drops (FIGS. 7A-7D). These results suggest that diabetes produces apoptosis in some cells through the caspase 3 pathway, which can be inhibited by beta-adrenergic receptor agonists.

Based upon the cell culture work with Compound 2, as described above, strong evidence exists for examining a beta-adrenergic receptor agonist in vivo for non-proliferative diabetic retinopathy. Therefore, a trial of 50 mM Compound 2 eye drops given 1 time each day for 8 months was begun. It was found that treatment of diabetic rats with Compound 2 was effective in preventing the loss of retinal thickness and apoptosis of cells of the ganglion cell layer that can occur in diabetic rodents as an acute response to the disease.

One of the key vascular findings common to diabetic retinopathy is the formation of degenerate capillaries, occurring at about 6 months of diabetes. Treatment with 1 mM Compound 2 was able to significantly reduce numbers of degenerate capillaries to levels similar to controls. In addition to reducing the loss of cells in the central retina following diabetes, eye drop therapy also was effective in reducing TNFα concentration throughout the treatment regimen, although it is most effective in the earlier time frames of the disease.

Diabetes produces a significant decrease in insulin receptor phosphorylation, which was increased by Compound 2 eye drops in vivo. For these experiments, the retina lysates from the diabetic rats at 2 and 8 months following 1 mM treatment with Compound 2 were used. In the studies of Compound 2 eye drops, the ERG was improved with eye drop treatment over the entire time frame, although the total amplitude of all three groups did decline over the 8-month period.

Similar to studies on prevention of loss of retinal thickness and cell number in diabetic rats, Compound 2 could reverse diabetic-like changes in the retina. As noted in FIGS. 2D-2F, 6 month diabetic rats receiving Compound 2 therapy for 2 months had improved retinal function. This was associated with an almost 50% increase in retinal thickness over diabetic rats alone and no loss of cells in the ganglion cell layer. The present invention suggests that BAR agonists can reverse damage from diabetes both functionally and histologically.

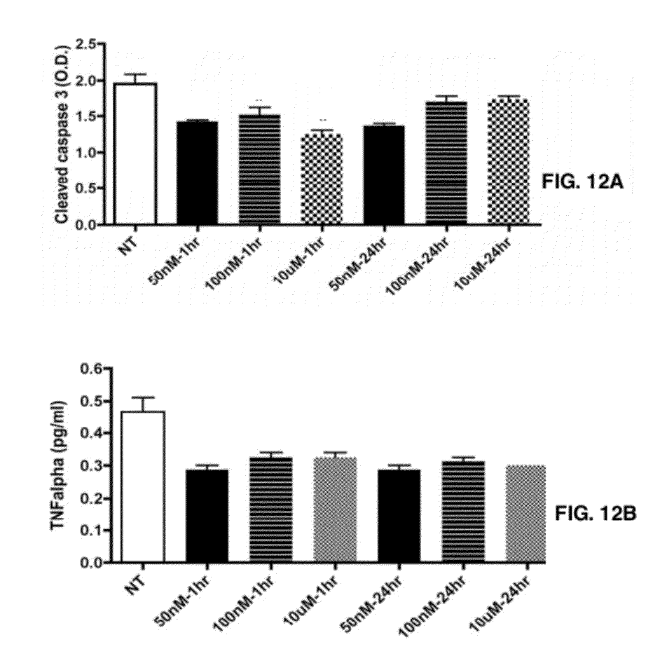

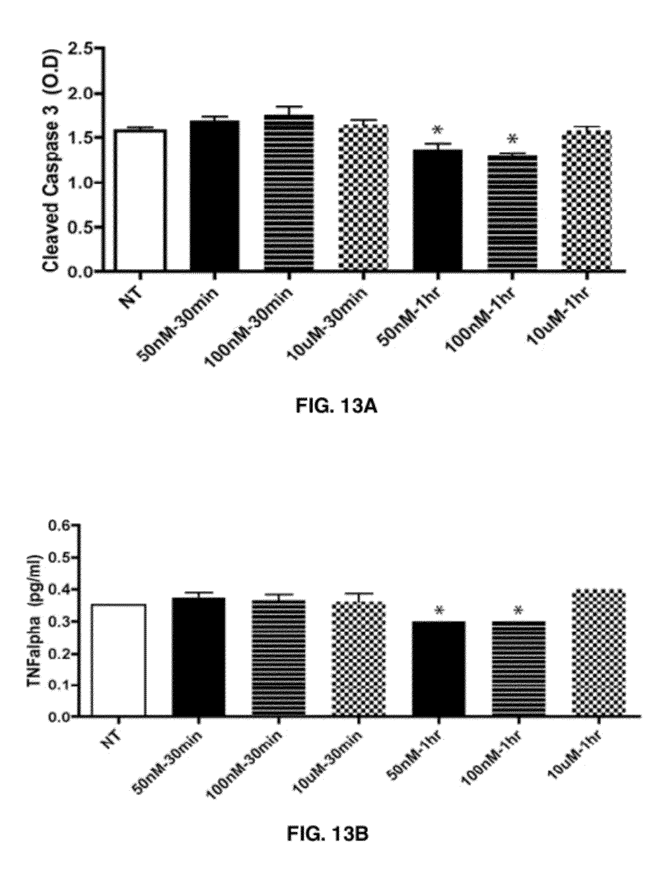

Isoproterenol can decrease cleaved caspase 3 levels in retinal endothelial cells (REC) and Müller cells cultured in hyperglycemic conditions and induce cardiovascular changes. Therefore the beta-adrenergic receptor agonist Compound 2 was developed. Caspase 3 is a pro-apoptotic protein cleavage of which and activation indicate cell death. Treatment with Compound 2 at the 50 nM concentration significantly decreased caspase 3 levels in Müller cells at the 24-hour time point (FIGS. 12A-2B, P<0.05 vs. NT-HG) and in REC (FIGS. 13A-13B, P<0.05 vs. Nt-HG) at the 30-minute time point. These results show that Compound 2 is able to decrease a key marker of cell apoptosis in vitro.

### Example 3

### In Vitro and In Vivo Effects of Compound 2 on TNFalpha

Compound 2 Prevents Apoptosis and TNFalpha Activation in REC and Müller Cells Cultured in Hyperglycemia

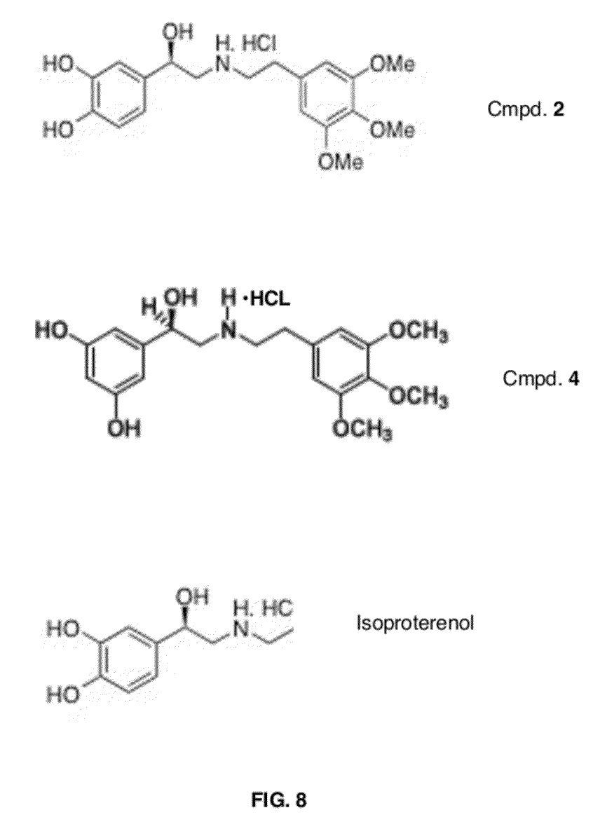

Compound 2 with beta-adrenergic receptor-like properties was developed, and its chemical name is (R)-4-[1-hydroxy-2-(1-ethylamino-3-,4-,5-trimethoxyphenyl)ethyl]benzene-1,2-diol hydrochloride and its chemical structure is shown and compared to isoproterenol in FIG. 8.

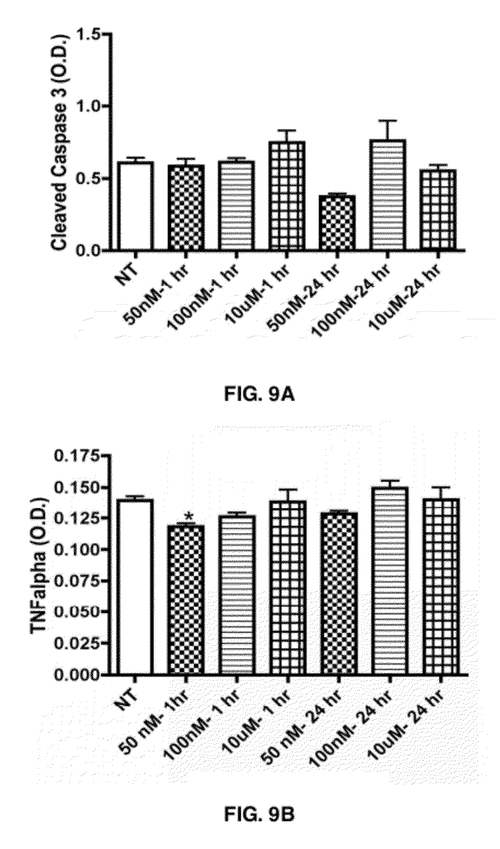

The ability of Compound 2 to prevent increased cleavage of caspase 3 and TNFalpha activity was investigated in cells cultured in high glucose. Inhibition of apoptosis and activation of inflammatory mediators significantly prevents both neuronal and vascular pathologies associated with diabetic retinopathy (7, 11-13). In Müller cells, blockade of apoptosis took 24 hours, while the inhibition of TNFalpha activity occurred much more quickly, within 1 hr when cells were treated with 10 μM isoproterenol. Very similar time courses for blockade of apoptosis and TNFalpha activity in Müller cells treated with Compound 2 were found, but at a significantly lower dose (50 nM Compound 2 vs. 10 μM isoproterenol). Compound 2 reduced TNFalpha levels by 19% and caspase 3 by 55% compared to not-treated cells (FIGS. 9A-9B).

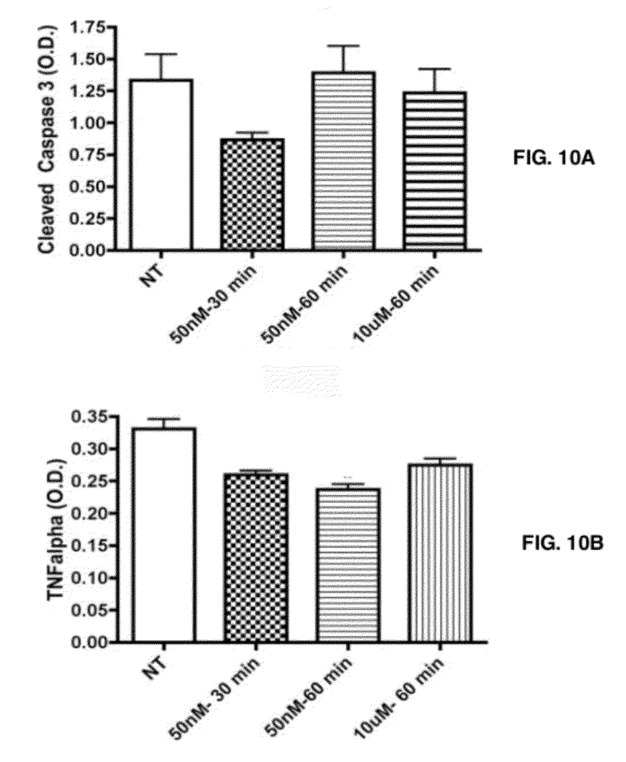

Similar to results in the Müller cells, Compound 2 significantly reduced the cleavage of caspase 3 and TNFalpha activity in retinal endothelial cells (REC) cultured in 25 mM glucose. Treatment with 50 nM Compound 2 significantly reduced caspase 3 levels by 54% and TNFalpha levels by 23% versus not-treated controls (FIGS. 10A-10B). Isoproterenol did not significantly reduce TNFalpha in retinal endothelial cells at 10 μM in vitro.

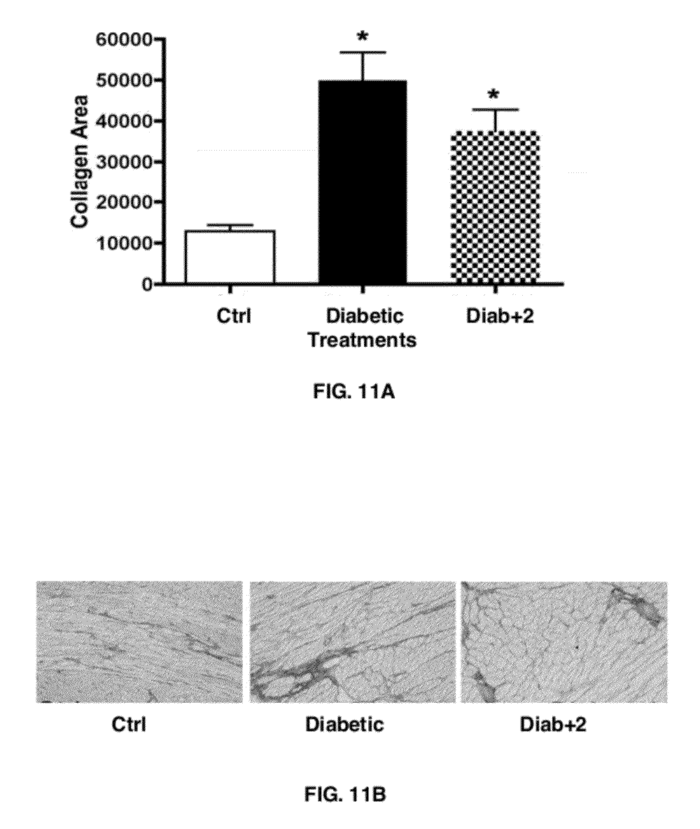

FIGS. 11A-11B shows the effect in type I diabetic rats treated daily with 1 mM Compound 2 (FIG. 11A). There was no difference in the left ventricle compared to untreated diabetic rats. Staining is for collagen intensity (FIG. 11B), which is increased in diabetes.

Compound 2 Reduced TNFalpha Levels and the Cleavage of Caspase 3 at a Dose Less than 10 μM

Human retinal endothelial cells (HREC) and rat Muller cells (rMC-1) in both glucose conditions were treated with Compound 2 at doses of 10 nM, 50 nM, 100 nM, 1 μM, and 10 μM of Compound 2. Cells of each type were grown in medium containing L-glucose to control for changes in osmolarity. 10 μM isoproterenol was also used for each condition as a positive control. Müller cells were treated for 1 hour and 24 hours, while HREC were treated for 30 and 60 minutes.

Following treatment, cells were collected into lysis buffer containing protease and phosphatase inhibitors. ELISA analyses for TNFalpha, cleaved caspase 3, and PKA were done according to manufacturers instructions. Data is compared against non-treated cells and cells at the various doses. A Kruskal-Wallis test is done, with a Dunn's test for secondary analyses. Compound 2 should significantly decrease TNFalpha levels and the cleavage of caspase 3 at a dose less than 10 μM (required for isoproterenol). The dose that decreases TNFalpha and caspase 3 levels also increases PKA activity.

Compound 2 Reduces TNFalpha and Caspase 3 Levels through PKA Activation

HREC and Müller cells were cultured as described in Example 1. Following serum starvation, cells were treated with 1 μM KT5720 to inhibit PKA activity for 30 minutes. Cells were then treated with the optimal dose of Compound 2 for 1 and 24 hours for Müller cells and 30 and 60 minutes for HREC. At the appropriate time after stimulation, cells were collected and processed for TNFalpha and caspase 3 ELISA analyses according to manufacturers instructions. A PKA ELISA also was done to ensure that PKA was properly inhibited. In addition to the treated cells, some cells were serum starved and received no treatment as a control. Additional dishes of each cell type were treated with KT5720 alone to insure that the PKA inhibitor alone had no effect on the cells, which would confound the data. Data was compared against non-treated cells and cells at the various doses. A Kruskal-Wallis test was performed, with a Dunn's test for secondary analyses. P<0.05 was accepted as significant.

### Compound 2 Effective In Vivo on Retinal Damage in Diabetic Rats

Rats were made diabetic with STZ injection. One week after STZ injection, daily topical Compound 2 was given at 1 mM. ERG was measured after 6 weeks. At 1 mM Compound 2 inhibited the loss of B-wave amplitude which occurred in diabetes.

Activated PKA Levels in the Retina after Administration of Compound 2

It was expected that topical delivery of Compound 2 would: 1) reach the retina and activate PKA at a lower dose than subconjunctival delivery or systemic (intravenous) delivery, and 2) produce fewer negative side effects, such as increased cardiovascular hypertrophy and physiological blood pressure.

B/PK was characterized following intravenous administration to rats. Following drug administration, blood samples (2-300 μL) were withdrawn from the jugular vein catheter at regular intervals after dosing (5, 15, 30, 45 minutes and 1, 2, 4, 8, 16, and 24 hours) into K3-EDTA anti-coagulated sampling tubes. Plasma was obtained by centrifugation and stored at −80° C. until analysis. The heart, lung and spleen were harvested in order to determine the tissue distribution of Compound 2. Cumulative urinary samples were collected over 24 hours post-dose and stored at −80° C. until analysis.

B/PK was characterized following topical administration. Following drug administration to rats, eyes were enucleated, irrigated with saline, and vitreous humor aspirated from the inner region of the vitreous chamber using an 18-gauge needle. Blood samples were obtained simultaneously in order to determine the extent of systemic exposure following topical administration. Vitreous humor and blood samples were obtained at regular intervals after dosing. Time points were determined based upon concentration time profiles obtained following intravenous administration of Compound 2.

For data analyses B/PK parameters were derived from the obtained plasma, vitreous humor, urine and feces concentration data by standard non-compartmental analysis. Terminal half-life was determined as the ratio of ln 2 divided by λz, the negative of the slope of the linear regression of the natural log concentration vs. time profile during the terminal phase. Systemic and ocular exposure was determined as plasma or ocular area under the concentration-time curve (AUC) using the trapezoidal rule. Compound 2 concentration determination and metabolite profiling is conducted using a validated LC/MS/MS assay based on the methodology described (14-15). The LC-MS/MS system comprises a Shimadzu HPLC system (Kyoto, Japan), API-4000 Q Trap tandem mass spectrometer (Foster City, Calif., USA) equipped with a turbo ion-spray and a HTC-PAL autosampler (Leap Technologies, Carrboro, N.C., USA).

### Compound 2 Decreased Insulin Receptor Signaling Inhibition

Rat Müller cells (rMC-1) were cultured in DMEM medium grown in normal glucose (low, 5 mM), diabetic levels of glucose (medium, 15 mM) and very high glucose (high, 25 mM) conditions. Medium was supplemented with 10% FBS and antibiotics. Five dishes of each type of cells were cultured in low, medium and high glucose alone and serve as non-treated controls. Five dishes at each glucose levels and each treatment were used at each time point using L-glucose as a control for osmolarity. Five dishes in each glucose condition also were treated with 10 nM insulin to serve as a positive control. The following treatments are assessed in rMC-1 cells and conditioned medium in all 3 glucose conditions.

Compound 2 stimulates beta-adrenergic receptors. It was determined whether Compound 2 alone could increase phosphorylation of insulin receptor tyrosine alone. Five dishes of each cell type were assessed using 50 nM Compound 2 (or optimal dose if different) at 30 min, 1, 2, 6, and 12 hours. After cells have been treated with Compound 2 for the appropriate time, cells were treated with lysis buffer containing phosphatase and protease inhibitors. Following a protein assay, samples were examined by Western blotting for phosphorylated insulin receptor (Tyr 1150/1151), phosphorylated ERK/12 (Tyr 44/42), Akt (Ser 473), and phosphorylated PI3K (p85^{Tyr458}, p55Tyr199). Western blot analyses of total protein levels of each protein were used to determine the ratio of phosphorylated protein to total protein.

It was determined also whether Compound 2 would activate PKA to elicit increased tyrosine phosphorylation of the insulin receptor and downstream intermediates. rMC-1 cells were cultured in the 3 glucose conditions described above. Following serum starvation, cells were treated for 30 minutes with 1 μM KT5720, a specific PKA inhibitor. After 30 minutes of KT5720 treatment, 50 nM Compound 2 was added for an additional 60 or 180 minutes. Some cells received the KT5720 treatment alone to ensure that treatment with this inhibitor had no effect on insulin receptor phosphorylation. L-glucose and no-treatment controls also were used. Additionally, site-directed mutagenesis as described above was done prior to treatment. Once collected, analyses were performed as described above. Additionally, a PKA ELISA was performed to ensure that no PKA activity was present.

### TNFalpha was a Key Intermediate in Compound 2 Regulation of Insulin Receptor Phosphorylation

Rat Müller cells (rMC-1) cells were used for these experiments and cultured in the 3 glucose conditions described above. For all treatments, five dishes of each cell type were used at the appropriate dose and time point using L-glucose controls for osmolarity. Five dishes for each experiment were used as non-treated controls. Following serum starvation, a number of treatments were applied to the culture medium.

Comparison of the TNFalpha Stimulation Alone and with Compound 2

Ser 307 phosphorylation on IRS-1, insulin receptor tyrosine phosphorylation, and Akt phosphorylation on serine 473 were measured to assess whether TNFalpha inhibits insulin signal transduction through IRS-1 and to determine the role of PKA in TNFalpha activities. rMC-1 cells were grown following the same protocol for glucose conditions as described above. Once the cells were starved, 5 dishes were treated with TNFalpha alone at 5 ng/ml for 30 minutes; 5 dishes were treated with TNFalpha for 30 minutes, followed by 50 nM Compound 2 for 60 minutes and 5 dishes were treated with TNFα and KT5720 for 30 minutes, followed by Compound 2 for 60 minutes. Following treatments, cells were processed as above, except that for assessment of insulin receptor substrate 1 (IRS-1), the focus was on serine 307, as this is the key site for TNFalpha blockade of insulin signal transduction (16). Site-directed mutagenesis was performed to convert Serine 307 to an alanine on IRS-1 to determine whether TNFalpha regulates insulin receptor responsiveness through this site in retinal Müller cells.

ELISA analyses were done to measure TNFalpha activity and cleaved caspase 3. TUNEL labeling was done to localize apoptotic cells. Insulin levels were measured in the cells and in the medium to measure whether TNFalpha stimulation regulates insulin production or secretion. For all treatments, mean densitometry values were obtained using the Kodak 2.0 software. The ratio of phosphorylated protein was compared to levels of total protein. A minimum of 4 independent experiments was done for each treatment group. Analysis of ELISA data was done using the manufacturers recommendation based on the standard curve generated in the assay. Statistical analyses were done using Kruskal-Wallis analyses, followed by Dunn's post-hoc test for all columns using Prism software. Analyses were done to compare treatments to non-treated groups. P<0.05 was accepted as significant.

Compound 2 Prevented and/or Reversed the Long-Term Neuronal and Vascular Alterations Common to Diabetic Retinopathy

The ability of Compound 2 to both prevent and reverse the retinal changes that occur in rodent pre-proliferative diabetic retinopathy was shown. 30 control rats, 30 diabetic rats, and 30 rats for Compound 2 were used. On day 0, 60 rats (30 diabetic, 30 Compound 2 were injected with 60 mg/kg of streptozotocin to eliminate insulin production by their pancreatic beta cells. Two days following streptozotocin injections, glucose measurements were obtained on all rats, with diabetes being accepted as glucose levels over 250 mg/dl. Beginning the day of initial glucose screening, eye drop therapy of 1 mM Compound 2 begins on 30 rats. The 30 rats that did not receive streptozotocin serve as control rats. Glucose levels were measured biweekly.

Analyses on the rats were done for acute changes (8 weeks of diabetes, 45 rats) and chronic changes (8 months of diabetes, 45 rats). In addition to all measurements on the retina, tissue sections of the heart were taken at 8 weeks and 8 months to insure that there is no hypertrophy of the ventricles due to the drug. A Western blot for myosin light chain, a surrogate marker of cardiovascular hypertrophy was also performed. Each month, all rats receive 2 analyses of visual function, electroretinogram (ERG) and live retinal imaging using Optical Coherence Topography (OCT). Retinal thickness and live retinal imaging were assessed using rodent OCT. This technology allows for non-invasive visualization of each layer of the retina for multiple testing of the same animal.

The optimal dose and time course of Compound 2 that was determined above was used. Thirty control rats, 30 diabetic rats, and 30 rats for Compound 2. On day 0, 60 rats (30 diabetic, 30 Compound 2 were injected with 60 mg/kg of streptozotocin to eliminate insulin production by their pancreatic beta cells. Two days following streptozotocin injections, glucose measurements were obtained on all rats, with diabetes being accepted as glucose levels over 250 mg/dl. Beginning the day of initial glucose screening, eye drop therapy of 1 mM Compound 2 begins on 30 rats. The 30 rats that did not receive streptozotocin serve as control rats. Glucose levels were measured biweekly.

Analyses on the rats were done for acute changes (8 weeks of diabetes, 45 rats) and chronic changes (8 months of diabetes, 45 rats). In addition to all measurements on the retina, tissue sections of the heart were taken at 8 weeks and 8 months to insure that there was no hypertrophy of the ventricles due to the drug. Myosin light chains as surrogate markers of cardiovascular hypertrophy were examined using Western blots. Each month, all rats were tested for visual function: electroretinogram (ERG) and live retinal imaging were performed using Optical Coherence Topography (OCT). For the ERG analyses, experiments were done according to described methods (2). While the animals were asleep for ERG analyses, blood pressure and pulse were monitored to detect potential negative cardiovascular events. At the 50 mM topical dose of isoproterenol these events have not been observed, so they were unlikely to happen when using Compound 2.

Retinal thickness and live retinal imaging were assessed using rodent OCT. Each layer of the retina was examined multiple times on the same animal in a non-invasive way to determine changing in specific regions over the course of the experiment. This data was combined with the histological measurements of retinal thickness. Light microscopy was used for histological examination of neuronal changes, which often occur in the acute phases. Formalin-fixed paraffin sections were stained with toluidine blue for light microscopy and morphometry of retinal thickness. Pictures were taken at four locations in the retina (both sides of the optic nerve and mid-retina) at 400×. The nuclei in the retinal ganglion cell layer (GCL) were counted in a 100 μm section of each picture. The thickness of the inner retina from the top of the inner nuclear layer to the inner limiting membrane was assessed using a Retiga camera attached to a Nikon Biophot light microscope with Qcapture software (Qlmaging, Burbay, BC, Canada). Retinal thickness and number of cells in the retinal ganglion cell layer were measured using OpenLab software (Improvision, Lexington, Mass.). Unpaired T-tests were used to compare data from control, diabetic, and diabetic+eye drop treated animals, with P<0.05 being accepted as significant.

The analyses of the retinal vasculature were based on published methods (6-7). For inflammatory analyses, whole retinal lysates were collected into lysis buffer containing protease and phosphatase inhibitors. A BCA protein assay was performed to determine protein content of the lysates. Luminex multi-plex cytokine analyses were performed to evaluate whether Compound 2 could significantly decrease protein activities of key inflammatory mediators in vivo. To analyze insulin receptor signaling pathways, retinal lysates from the control, diabetic, and diabetic+Compound 2 animals were collected and assessed.

### Compound 2 Reversed Retinal Damage Due to 6 Months-Old Untreated Induced Diabetes

Ninety rats were used (30 eye drop without diabetes, 30 with diabetes alone, 30 with topical Compound 2 following 6 months of untreated diabetes). Streptozotocin was used in the dose of 60 mg/kg on day 0 and 60 to induce diabetes in rats. The criterium for the experimental diabetes development was blood glucose level of over 250 mg/dl two days after treatment with Streptozotocin. The last group of animals was subjected to topical eye drop therapy using Compound 2 at 1 mM. The remaining 30 rats served as pure diabetic controls. Acute (8 weeks after initiation of eye drops, 45 rats) and chronic changes (8 months following initiation of eye drop therapy, 45 rats) using the same measurements as described.

Compound 4 Prevented Apoptosis and TNFalpha Activation in REC and Müller Cells Cultured in Hyperglycemia

FIG. 8 depicts the chemical structure of Compound 4,5-(1-hydroxy-2-[2-(3,4,5-trimethoxy-phenyl)-ethylamino]-ethyl)-benzene-1,3-diol hydrochloride. Muller cells and retinal endothelial cells (REC) cells were cultured and treated with 50 nM Compound 4 as per the protocol for Compound 2 in Example 3. ELISA data show that Compound 4 has more beta-2-adrenergic receptor activity in Muller cells and REC cells, which means likely less heart effects, and works at 50 nM in vitro to reduce TNFalpha levels and the cleavage of caspase 3. Compound 4 is more effective and works faster than Compound 2 in Muller cells, likely because the dominant beta-adrenergic receptor in Muller cells is the beta-2-adrenergic receptor subtype. In retinal endothelial cells, Compound 4 does take longer to activate response retinal endothelial cells, likely due to less beta-1-adrenergic receptor activity, which is the dominant receptor in retinal endothelial cells.

### Effects of Isomers of Compound 2 on TNFalpha Concentration

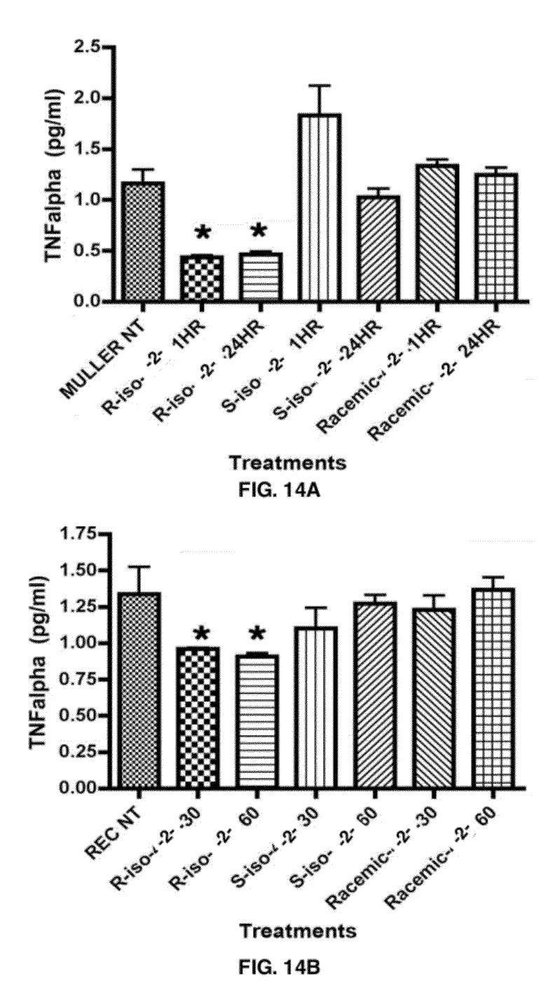

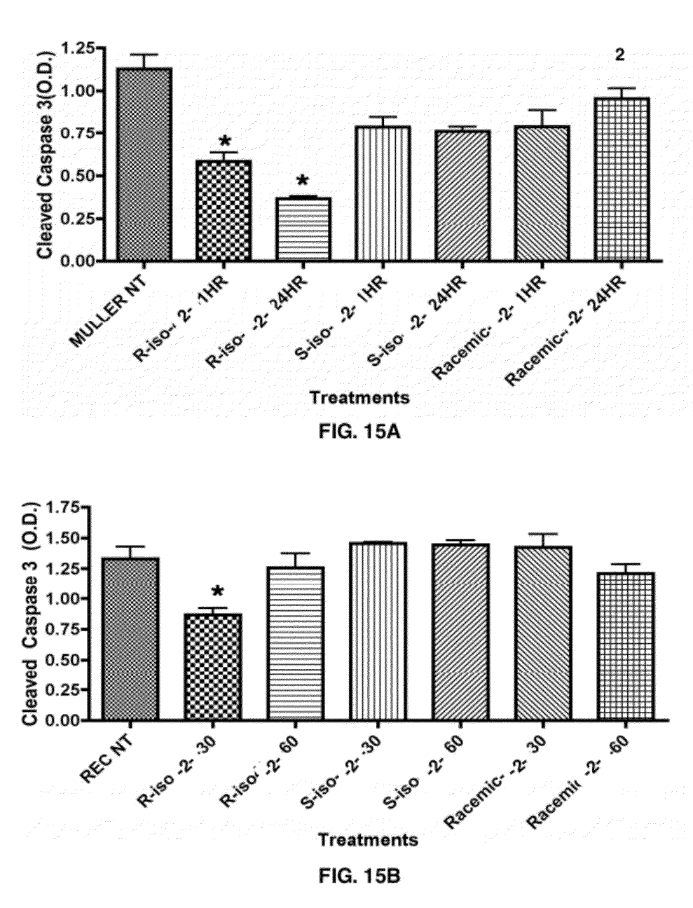

FIGS. 14A-14B show the effects the R-isomer of Compound 2, the S-isomer of Compound 2 and racemic Compound 2 at either 1 hour or 24 hours of treatment on TNFalpha concentration in Muller and retinal endothelial cells. FIGS. 15A-15B shows the effects the R-isomer of Compound 2, the S-isomer of Compound 2 and racemic Compound 2 at either 1 hour or 24 hours of treatment on cleaved caspase 3. This data clearly shows that the R-isomer of Compound 2 is superior to the S-isomer of Compound 2 and racemic Compound 2.

### Example 4

### Biopharmaceutic/Pharmacokinetic (B/PK) Parameters at 10× Therapeutic Doses of Compound 2 in Rat Model

Since an effective topical dose in rats of 1 mg/kg did not result in detectable Compound 2 in plasma, the dosing was increased to 10× the effective dose to attempt to detect Compound 2 in the plasma and tissues.

### Detection of Compound 2 in the Plasma

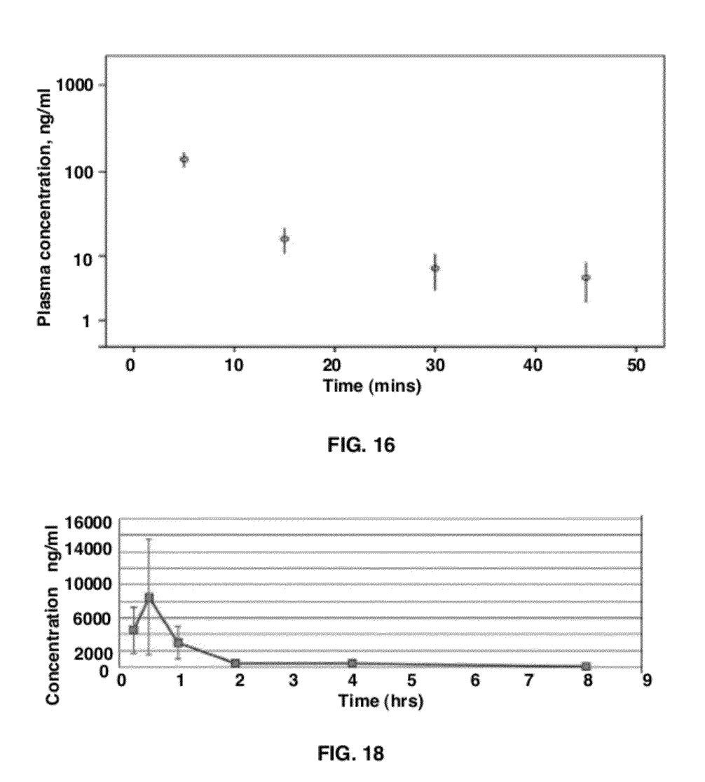

Compound 2 was delivered topically and intravenously to the rats in a therapeutic dose of 1 mM or 1 mg/kg. Topical delivery of Compound 2 at a therapeutic dose was below the limits of detection. Animals receiving Compound 2 intravenously had a rapid clearance of the drug (<1 hour) (FIG. 16).

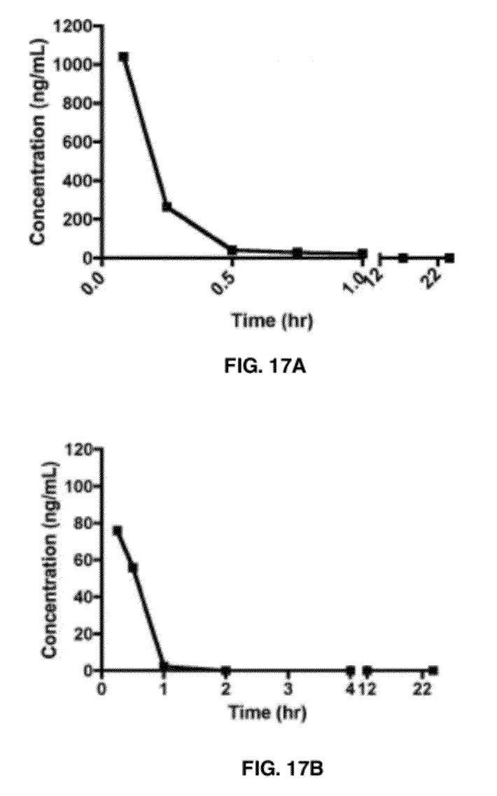

Because Compound 2 was not detected in the plasma following topical delivery at the 1 mg/kg therapeutic dose, the dose was increased to 10 mg/kg and plasma levels were assessed plasma following both topical and intravenous delivery of Compound 2. Despite the increase in dosing to 10×, Compound 2 still was not detected in the plasma of topically treated animals except at only 2 of the time points tested, i.e., at a concentration of ˜80 ng/ml (FIG. 17B). Analyses were done at 10 time points from 0.8 hr to 24 hr. Intravenous delivery showed ˜1 ug/mL clearing within 30 minutes (FIG. 17A). Compound 2 is rapidly removed from the plasma whether administered intravenously or topically at a dose 10× therapeutic dose. This strongly suggests that Compound 2 does not enter the systemic circulation, thus further decreasing changes of deleterious side effects.

### Detection of Compound 2 in the Vitreous Humor, Heart, Lung, Spleen

Since the vitreous humor can serve as a reservoir of drugs that bathe the retina, the concentration of Compound 2 in the vitreous humor was evaluated after topical delivery of 10 mg/kg of Compound 2. Compound 2 concentrations in the vitreous humor peak at <1 hour and all detectable levels of Compound 2 are gone by 2 hours following topical application (FIG. 18). The vitreous humor collects approximately 8 ug/ml of topical Compound 2 within 1 hour, however no Compound 2 is detected in the vitreous humor after 2 hours.

Compound 2 was delivered at a concentration 10× the therapeutic dose to detect levels of the compound in the heart, lung and spleen. Table 2 lists the concentrations of Compound 2 detected in the heart, lung, and spleen after 24 hours following topical delivery of 10 mg/kg Compound 2. Compound 2 is present in very low levels in the heart and lung, with slightly higher levels in the spleen.

TABLE 2

| | Animal 1 | Animal 2 | Animal 3 | Animal 4 | Animal 5 |
|---|---|---|---|---|---|
| | ng/g | ng/g | ng/g | ng/g | ng/g |
| Heart | <400 | <400 ng/g | <400 ng/g | <400 ng/g | <400 ng/g |
| Lung | 463.43 | 2262.08 | 2862.22 | 2060.08 | 535.24 |
| Spleen | 12134.03 | 8393.8 | 7061.2 | 4544.4 | 34331.0 |

### Example 5

### Beta-Adrenergic Receptor Pharmacology/Pharmacokinetics of Compound 2 in a Dog

Without being limited by theory, it is contemplated that retinal endothelial cells, are critical in the cellular mechanisms of action of Compound 2 in the diabetic retina of humans. The dog is an accepted diabetic model for human diabetes. The dog model is utilized to verify the binding affinity of Compound 2 to beta-adrenergic receptors, as well as its ability to induce cAMP accumulation. These are critical pharmacology studies of a novel drug to determine the optimal binding kinetics in retinal endothelial cells, as we feel these cells are critical in the cellular mechanisms of action of Compound 2 in the diabetic retina.

### Example 6

### Effect of Compound 2 on Proliferative Diabetic Retinopathy

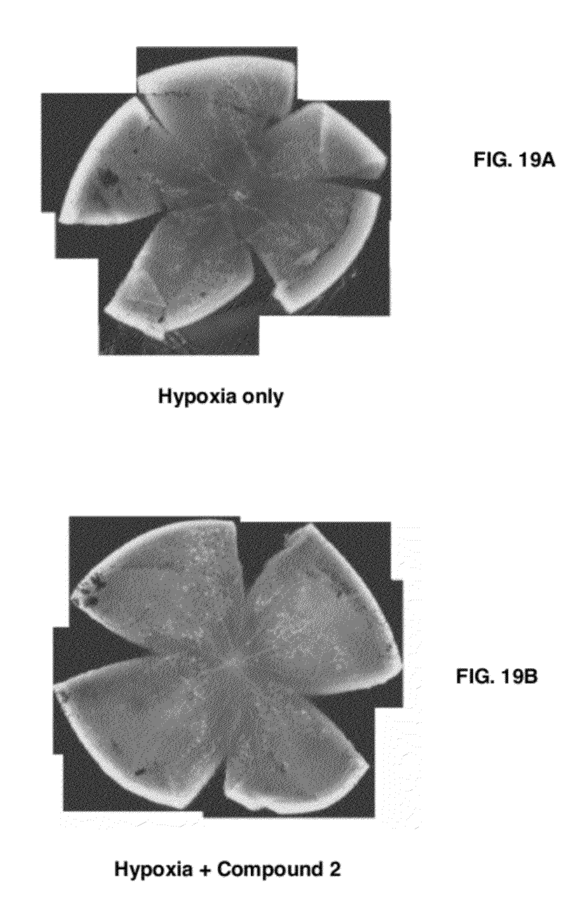

A model of oxygen-induced retinopathy was used to determine the effects in vivo of Compound 2 on mice. Mice were placed into a chamber with 100% oxygen at day 7 of life. On day 12, they were removed from the chamber into normal air. The retina interprets this as hypoxia and starts angiogenesis. Mice were treated with 1 mM Compound 2 in eye drops once daily on days 13, 14, 15. Animals were sacrificed on day 17. FIGS. 19A-19 Retinal flat mounts were prepared. Mice that did not receive Compound 2 (FIG. 19A) had a very under-developed retinal vasculature. In Compound 2 treated mice (FIG. 19B), the retina is much more developed and appears normal. This demonstrates a therapeutic effect of Compound 2 against angiogenesis in an in vivo model of proliferative diabetic retinopathy.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. Also, the terms in the claims have their plain, ordinary meaning unless otherwise explicitly and clearly defined by the patentee.

The following references are cited herein.
1. Phipps et al., Invest Ophthalmol V is Sci, 2007, 48(2):927-34.
2. Jiang, Y. and J. J. Steinle, Invest Ophthalmol V is Sci, 2009.
3. Forte et al. J Neurosci Methods, 2007; 169:191-200.
4. Weymouth, A. E. and Vingrys, A. J., Prog Retin Eye Res, 2008, 27:1-44.
5. Kern, T. S, and Engerman, R. L., Curr Eye Res, 1994, 13:863-867.
6. Kowluru et al. Diabetes, 2001, 56:373-379.
7. Mizutani et al. J Clin Invest, 1996, 97:2883-2890.
8. Steinle et al. Exp Eye Res, 2009, 88:1014-1019.
9. Steinle et al. Exp Eye Res, 2008, 87:30-34.
10. Reiter et al. Diabetes, 2006, 55:1148-1156.
11. Walker et al. Invest Ophthalmol V is Sci, 2007, 48(11):5276-81.
12. Joussen et al. Faseb J, 2004, 18(12):1450-1452.
13. Williams, K. P. and Steinle, J. J., Exp Eye Res, 2009, 89(4):448-455.
14. Trester-Zedlitz et al. Biochemistry, 2005, 44(16):6133-4613.
15. Gu et al. Anal Chim Acta, 2008, 609(2):192-200.
16. Boura-Halfon et al. Am J Physiol Endocrinol Metab, 2009, 296(4):E581-91.

Any patents or publications mentioned in this specification are indicative of the level of those skilled in the art. Further, these patents and publications are incorporated by reference herein to the same extent as if each publication was specifically and incorporated by reference. One skilled in the art would appreciate that the present invention is well adapted to carry out the objects and obtain the ends mentioned, as well as those objects, and advantages inherent herein. Changes therein and other uses which are encompassed within the spirit of the invention as defined by the scope of the claims will occur to those skilled in the art.

What is claimed is:

1. A method for improving function in a retinal cell associated with a diabetic condition, comprising:
contacting the cell with a beta-adrenergic receptor agonist, said beta-adrenergic receptor agonist increasing insulin signaling and decreasing TNFα-induced apoptosis, thereby improving the function in the retinal cell.

2. The method of claim 1, wherein the beta-adrenergic receptor agonist has the chemical structural formula: 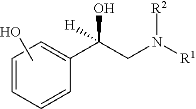
wherein R¹ is (CH₂)ₙ(CH₃)₂ or 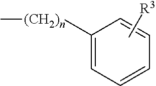
where n is 1 to 4;
R² is H or H.HX, where X is a halide; and
R³ is O(CH₂)ₘCH₃ at one or more of C2-C6, where m is 0 to 4.

3. The method of claim 2, wherein R¹ is (CH₂)ₙ(CH₃)₂ and R² is H.

4. The method of claim 3, wherein the beta-adrenergic receptor agonist is isoproterenol.

5. The method of claim 2, wherein R¹ is (CH₂)₂-phenyl, R² is H or H.HCl and R³ is O(CH₂)ₘCH₃ at C3, C4 and C5.

6. The method of claim 5, wherein the beta-adrenergic receptor agonist is (R)-4-[1-hydroxy-2-[3,4,5-trimethoxy-phenyl)-ethylamino]-ethyl)-benzene-1,2-diol, (R)-4-[1-hydroxy-2-[3,4,5-trimethoxy-phenyl)-ethylamino]-ethyl)-benzene-1,2-diol hydrochloride, (R)-5-(1-hydroxy-2-[2-(3,4,5-trimethosy-phenyl)-ethylamino]-ethyl)-benzene-1,3-diol, (R)-5-(1-hydroxy-2-[2-(3,4,5-trimethoxy-phenyl)-ethylamino]-ethyl)-benzene-1,3-diol hydrochloride or an R-isomer thereof.

7. The method of claim 1, wherein the retinal cell is contacted in vitro or in vivo.

8. The method of claim 1, wherein the diabetic condition is diabetic retinopathy, preproliferative diabetic retinopathy, proliferative diabetic retinopathy, or other hyperglycemic conditions.

9. A method for treating a diabetic retinopathic condition in a subject, comprising:
administering one or more times a pharmacologically effect amount of one or more beta-adrenergic receptor agonists to the subject, wherein said agonist improves retinal cell function, thereby treating the diabetic retinopathy.

10. The method of claim 9, wherein the beta-adrenergic receptor agonist has the structural formula: 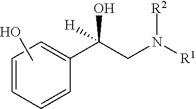
wherein R¹ is (CH₂)ₙ(CH₃)₂ or 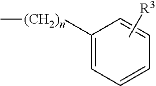
where n is 1 to 4;
R² is H or H.HX, where X is a halide; and
R³ is O(CH₂)ₘCH₃ at one or more of C2-C6, where m is 0 to 4.

11. The method of claim 10, wherein R¹ is (CH₂)ₙ(CH₃)₂ and R² is H.

12. The method of claim 11, wherein the beta-adrenergic receptor agonist is isoproterenol.

13. The method of claim 10, wherein R¹ is (CH₂)₂-phenyl, R² is H or H.HCl and R³ is O(CH₂)ₘCH₃ at C3, C4 and C5.

14. The method of claim 13, wherein the beta-adrenergic receptor agonist is (R)-4-[1-hydroxy-2-[3,4,5-trimethoxy-phenyl)-ethylamino]-ethyl)-benzene-1,2-diol, (R)-4-[1-hydroxy-2-[3,4,5-trimethoxy-phenyl)-ethylamino]-ethyl)-benzene-1,2-diol hydrochloride, (R)-5-(1-hydroxy-2-[2-(3,4,5-trimethosy-phenyl)-ethylamino]-ethyl)-benzene-1,3-diol, or (R)-5-(1-hydroxy-2-[2-(3,4,5-trimethoxy-phenyl)-ethylamino]-ethyl)-benzene-1,3-diol hydrochloride.

15. The method of claim 9, further comprising:
administering one or more other diabetic or retinopathic drugs to the subject.

16. The method of claim 15, wherein the other drugs are administered concurrently or sequentially with the beta-adrenergic receptor agonist(s).

17. The method of claim 9, wherein the beta-adrenergic receptor agonist comprises a pharmaceutical composition with a pharmaceutically acceptable carrier.

18. The method of claim 17, wherein the pharmaceutical composition is suitable for topical, subconjunctival or intravenous administration.

19. The method of claim 9, wherein the diabetic retinopathic condition is preproliferative retinopathy or proliferative diabetic retinopathy.

20. A beta adrenergic receptor agonist having the chemical structural formula: 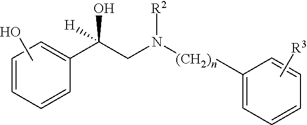
wherein n is 1 to 4;
R² is H or H.HX, where X is a halide; and
R³ is O(CH₂)ₘCH₃ at one or more of C2-C6, where m is 0 to 4.

21. The beta adrenergic receptor agonist of claim 20, wherein n is 2, R² is H or H.HCl and R³ is OCH₃ at C3, C4 and C5.

22. The beta adrenergic receptor agonist of claim 21, wherein said beta adrenergic receptor agonist is (R)-4-[1-hydroxy-2-[3,4,5-trimethoxy-phenyl)-ethylamino]-ethyl)-benzene-1,2-diol, (R)-4-[1-hydroxy-2-[3,4,5-trimethoxy-phenyl)-ethylamino]-ethyl)-benzene-1,2-diol hydrochloride, (R)-5-(1-hydroxy-2-[2-(3,4,5-trimethoxy-phenyl)-ethylamino]-ethyl)-benzene-1,3-diol, (R)-5-(1-hydroxy-2-[2-(3,4,5-trimethoxy-phenyl)-ethylamino]-ethyl)-benzene-1,3-diol hydrochloride.

23. The agonist of claim 20, wherein said structure is in R-isomeric form.

24. A pharmaceutical composition comprising the beta-adrenergic receptor agonist of claim 20 and a pharmaceutically acceptable carrier.
